(19) United States
(12) Patent Application Publication
(10) Pub. No.: US 2024/0358671 A1
(43) Pub. Date: Oct. 31, 2024

(54) COMPOSITIONS AND METHODS TO TREAT SKIN DISORDERS

(71) Applicant: THE TRUSTEES OF INDIANA UNIVERSITY, Bloomington, IN (US)

(72) Inventors: Chandan K. SEN, Indianapolis, IN (US); Sashwati ROY, Indianapolis, IN (US); Mithun SINHA, Indianapolis, IN (US); Nandini GHOSH, Indianapolis, IN (US)

(21) Appl. No.: 18/687,205

(22) PCT Filed: Sep. 7, 2022
(86) PCT No.: PCT/US2022/042753

§ 371 Date: Feb. 27, 2024

Related U.S. Application Data
(60) Provisional application No. 63/242,059, filed on Sep. 9, 2021.

(51) Int. Cl. A61K 31/343; A61K 31/164; A61P 17/02; A61P 31/04

(52) U.S. Cl. CPC A61K 31/343; A61K 31/164; A61P 17/02; A61P 31/04

(57) ABSTRACT

Compositions and methods are disclosed for treating a skin condition in a mammal in need thereof by administering to the affected tissues a therapeutically effective amount of a composition comprising a bacterial ceramidase inhibitor and/or a ceramide.

### CROSS REFERENCE TO RELATED APPLICATIONS

This application claims priority to U.S. Provisional Patent Application Nos. 63/242,059 filed on Sep. 9, 2021, the disclosure of which is expressly incorporated herein.

### STATEMENT OF GOVERNMENT SUPPORT

This invention was made with government support under DK114718, DK125835, GM108014 and NS042617 awarded by National Institutes of Health. The government has certain rights in the invention.

### BACKGROUND

Skin acts as a natural barrier between internal and external environments and therefore plays an important role in vital biological functions such as protection against mechanical and chemical injury, microorganisms, and ultraviolet damage. Human skin is made up mainly of two main layers, the outer epidermis and the underlying dermis.

The epidermis covers the dermis and is in direct contact with the external environment. The dermis has the main role of protecting the body against the dehydration and external attack. Cells constituting the epidermis are delimited by a lipid domain. In the course of differentiation, phospholipids, the role of which consists in producing the fluid structure of the cell membranes of the living layers of the epidermis, are gradually replaced by a mixture composed predominantly of fatty acids, cholesterol and ceramides (sphingolipids). These lipids are responsible for the “barrier” properties of the epidermis, particularly of the outermost layer of the epidermis, the stratum corneum.

Epidermal lipids are mainly synthesized in living epidermis. They are made up mainly of phospholipids, sphingolipids, cholesterol, free fatty acids, and triglycerides. Ceramides are a class of sphingolipids that play a paramount role in cellular signaling and are linked to cell proliferation, differentiation and apoptosis in human epidermis. Epidermal lipids are necessary for maintaining the multilamellar structure of the intercorneocytic lipids. They also contribute to the “barrier” function of the epidermis and for overcoming water loss/moisturization problems.

The present invention addresses problems associated with interactions between host skin lipids and bacterial factors capable of metabolizing them in the skin wound microenvironment. Such interaction are associated with impaired wound closure.

### SUMMARY

In one aspect, the disclosure relates to a compositions for treating skin conditions associated with compromised barrier function. More particular, the present disclosure is directed to skin conditions where barrier function is sub-optimal due to depletion of epidermal lipids including ceramides. In one embodiment the depletion of ceramides is the result of a pathogenic infection of the skin tissue, particularly in the case of dermal wounds. In one embodiment, tissues exhibiting compromised barrier function are treated with a pharmaceutical composition comprising a therapeutically effective amount of a bacterial ceramidase inhibitor and optionally one or more ceramides.

In another aspect, the disclosure relates to a method to treat a skin condition in a mammal in need thereof, by administering a therapeutically effective amount of a composition comprising a bacterial ceramidase inhibitor and/or a ceramide to the skin of the mammal in need thereof. In one embodiment the method comprises simultaneously administering one or more bacterial ceramidase inhibitor in conjunction with the administration of one or more ceramides. The bacterial ceramidase inhibitors and ceramides can be administered simultaneously or sequentially. When administered sequentially, the bacterial ceramidase inhibitors and ceramides are administered within a timeframe when the first administered compound is still active.

Additional embodiments, features, and advantages of the disclosure will be apparent from the following detailed description and through practice of the disclosure.

### BRIEF DESCRIPTION OF THE DRAWINGS

FIGS. 1A-1F illustrate that inducible bacterial ceramidases deplete host cutaneous ceramide. FIG. 1A: Schematic presentation of the timeline of porcine model of chronic wound biofilm infection. FIG. 1B: Induction of bacterial ceramidase (bcdase) in biofilm infected porcine wounds. Real-time qPCR analysis of bcdase expression spontaneously infected (SI) and wild type P. aeruginosa (PAwt) infected porcine wound epidermis (d7 post-infection) collected by laser capture micro-dissection (LCM). Data presented as mean±SEM, (n=5). FIG. 1C: PAΔcer biofilm was deficient in secreted bcdase activity. Data presented as mean±SEM, (n=6). See FIG. 8A for gene bcdase expression data. [NBD-CER-C12-NBD-Ceramide, NBD-DA-NBD-dodecanoic acid]. FIG. 1D: Depletion of ceramide in porcine wounds infected with PAwt biofilm compared to SI or wounds infected with ceramidase mutant of P. aeruginosa (PAΔcer). Data presented as mean 544±SEM, (n=6). FIG. 1E: Depletion of ceramides in porcine skin lipid exposed to PAwt, or PAΔcer as measured by LC/MS/MS targeted-lipididomic approach. Same skin lipid not exposed to any bacteria was used as sham control. Hierarchical Cluster Analysis revealed down-regulation of 19 species of ceramide in the skin lipid (n=6). Individual ceramides that were specifically affected are illustrated in FIGS. 9A-9S. FIG. 1F: Functional restoration of barrier function of cutaneous wounds, measured by trans-epidermal water loss (TEWL), was compromised following PAwt biofilm infection. Data presented as mean±SEM (n=6). ‡p<0.05 as compared to PAΔcer; and *p<0.05 as compared to SI.

FIGS. 2A-2C illustrate that host lipids facilitate PAwt biofilm aggregate formation.

Cutaneous lipids induced biofilm aggregate formation in an in vitro polycarbonate membrane biofilm system after 24 h of inoculation. Increased abundance of EPS in PAwt biofilm in response to host lipids was observed. FIG. 2A: Quantification of biofilm aggregates using WGA staining. Data presented as mean±SEM, n=10. FIGS. 2B and 2C: Host lipids induce biofilm gene expression in pathogen PAwt. Data presented as mean±SEM, (n=5).

FIGS. 3A-3J illustrates biogenesis of host long chain ceramides is compromised under conditions of PAwt biofilm infection. FIG. 3A: Long chain ceramide biogenesis pathways outlined. FIG. 3B: Downregulation of CerS3 in wound-site skin tissue infected with PAwt compared to SI or PAΔcer measured by qPCR. Data presented as mean±SEM (n=5-7). FIG. 3C: PAwt lowered the levels of the levels of dihydroceramide (C18DHCer) in skin lipids. Measured by LC/MS/MS targeted lipidomic approach. Data are presented as mean±SEM (n=6). FIG. 3D: Wound fluid collection from chronic wound patients who underwent negative pressure wound therapy (NPWT) as part of standard of care. FIG. 3E-3F: Lower levels of dihydroceramide in cyclic diGMP-rich human wound fluid. cyclic di-GMP is a marker of PA biofilm infection (n=5).

FIG. 3G: Biofilm induced miR-106b targets host CerS3. FIG. 3H: Elevated miR-106b in human keratinocytes (HK) infected with PAwt biofilm as measured by qPCR. Data presented as mean±SEM (n=5-6). miR-106b is predicted to target the 3′-UTR of CerS3 position 331-355 according to RNAHybrid™ algorithm. FIG. 3I: miR-106b silenced pmiR-Target-Cers-3′-UTR in human keratinocytes (HK). FL, Firefly luciferase; RL, Renilla luciferase. Data are mean±SEM (n=4). FIG. 3J: miR-106b silenced CerS3 protein expression in human keratinocytes (HK). Data are presented as mean±SEM (n=6).

FIG. 4 is a schematic drawing showing wound-site skin PPARδ expression is downregulated in response to PAwt biofilm infection.

FIG. 5 illustrates a mechanism related to PA biofilm formation that relies on the theft of host lipid factors which the bacteria use to turn on and to sustain its bolstered ceramidase system.

FIGS. 6A-6C show validation of PAΔcer for mutation of bcdase. bcdase was not detected in PAΔcer. Data presented as mean±SEM, (n=4). CFU count from the wounds of SI, PAwt or PAΔcer wounds for d7 and d14 post-infection. Data presented as mean±SEM, (n=4-6). Abundance of Pseudomonas aeruginosa (PA) among other bacterial species from the wound-site post-infection as measured through next generation sequencing (NGS) of 16S rRNA variable region. Data presented as mean±SEM, (n=4). Evidence of biofilm structure in wounds infected with PAwt or PAΔcer. FIG. 6D: Increased PA biofilm matrix in wounds infected with PAwt compared to SI or PAΔcer. Porcine d14 wound tissues were immuno-stained with PA biofilm matrix component specific pel (green) antibody and DAPI (blue). Data presented as mean±SEM, (n=5). FIG. 6E: Exposure to cutaneous lipids induced bcdase expression in PAwt but such induction is attenuated in PAwt exposed to depleted skin lipids. Data presented as mean±SEM, (n=4-6). FIG. 6F: Hypothetically, host cutaneous lipids induce bcdase expression in PAwt via upregulation of the Pseudomonas transcriptional activator SphR. FIG. 6G: Exposure to cutaneous lipids induced SphR expression in PAwt. Data presented as mean±SEM, =5-6).

FIGS. 7A-7U relate to ceramide depletion and ceramidastin rescue. FIGS. 7A-7S: Ceramide depletion, as measured by LC-MS/MS, in cutaneous lipids. Following exposure to bacteria as applicable, lipids were extracted and analyzed via a targeted approach. Data thus obtained were subjected to analysis by ANOVA using R3.5.3 to specifically identify those lipids with significant differences between the three groups. Those with significance as tested by ANOVA were subjected to post hoc analysis using Tukey-HSD with a FDR cutoff of 0.05. Lipids with group differences that passed these thresholds were illustrated as box plots. Skin ceramides have been denoted using the standard nomenclature following the format of fatty acyl type, sphingoid backbone type, and sphingoid backbone length-fatty acyl length. NS=Cer 2 (with non-hydroxy fatty acid, sphingoid backbone); NP=Cer 3 (non-hydroxy fatty acid, phytopshingoid backbone); NH=Cer 8 (non-hydroxy fatty acid, 6-hydroxy sphingoid backbone); NDS=Cer 10 (non-hydroxy fatty acid, dihydroxy sphingoid backbone); AS=Cer 5 (α-hydroxy fatty acid, sphingoid backbone); AP=Cer 6 (α-hydroxy fatty acid, phytosphingoid backbone). The Y-axis values are intensity in cps (counts per second). FIG. 7T: Human keratinocyte (HK) membrane ceramides were found to be sensitive to infection by PAwt but not to PAΔcer infected monolayer of keratinocytes based on immunostaining with anti-ceramide (red) antibody and DAPI (blue). FIG. 7U: Ceramidastin, a PA bcdase inhibitor, rescued depletion of keratinocyte ceramide in response to PAwt biofilm infection. Human keratinocytes (HK) were treated with conditioned media (25% v/v) of cells co-cultured with PA biofilm containing ceramidastin (10 μg/ml) for 24 h.

FIGS. 8A-8N illustrate ceramide depletion. FIGS. 8A-8L Ceramide depletion, as measured by LC-MS/MS, in cutaneous lipids exposed to biofilm-infected keratinocyte conditioned media (CM). As indicated, to test the significance of PA bcdase, the CM contained ceramidastin (CST). Lipids were extracted and analyzed using an untargeted approach as described in Materials and Methods. The resultant data were analyzed using R 3.3. Wilcoxon Rank-Sum test was used for all group analyses and Kruskal-Wallis followed by Benjamini Hochberg correction was performed for multiple comparisons with significance at p<0.05. Skin ceramides have been denoted using the standard nomenclature following the format of fatty acyl type, sphingoid backbone type, and sphingoid backbone length-fatty acyl length. NS=Cer 2 (with non-hydroxy fatty acid, sphingoid backbone); NP=Cer 3 (non-hydroxy fatty acid, phytopshingoid backbone); NH=Cer 8 (non-hydroxy fatty acid, 6-hydroxy sphingoid backbone); NDS=Cer 10 (non-hydroxy fatty acid, dihydroxy sphingoid backbone); AS=Cer 5 (α-hydroxy fatty acid, sphingoid backbone); AP=Cer 6 (α-hydroxy fatty acid, phytosphingoid backbone). The Y-axis values are in mol %. (M) Digital wound images and planimetry shown. Data presented as mean±SEM, (n=6). p<0.05 as compared to PAΔcer and *p<0.05 as compared to SI.

FIGS. 9A-9I illustrate the likely role of skin ceramide degradation products in biofilm formation. FIGS. 9A-9B: Depletion of cutaneous lipids as achieved by a methanol-chloroform method. Data represented as mean±SEM, n=5. Cutaneous lipids induced biofilm aggregate formation in an in vitro polycarbonate membrane biofilm system as determined by SEM. Large field shown. FIG. 9C: PAwt+vehicle (PBS); FIG. 9D, PAwt+skin lipids; FIG. 9E, PAwt+depleted lipids; FIG. 9F, PAΔcer+vehicle (PBS) FIG. 9G, PAΔcer+skin lipids. Scale bar=1 μm, 10,000× magnification. Yellow dots mark thick EPS formation. Yellow arrowheads recognize EPS fibers. Growth curve of PAwt or PAΔcer in LB media. FIG. 9H: Data presented mean±SEM, n=8. Growth curve of PAwt or PAΔcer in M9 minimal media supplemented with or without skin lipids. FIG. 9I: Data presented mean±SEM, n=12-16. *p<0.05 as compared to PAwt.

FIGS. 10A-10I illustrate additional experimental results. FIG. 10A: Downregulation of CerS3 in LCM captured PAwt infected wound epithelium compared to PAΔcer infected wound epithelium measured by qPCR; data presented as mean±SEM (n=5). FIGS. 10B-10G) Dihydroceramide levels in human chronic wound fluids negatively co-relate with cyclic diGMP marker of PAwt biofilm (n=5). FIG. 10H: Demographic characteristics of chronic wound patients. FIG. 10I: CerS3 3′UTR vector map.

FIGS. 11A-11O illustrate additional experimental results. FIG. 11A: Vector map of PPARδ promoter luciferase reporter. FIGS. 11B-11C: PPARδ drives CerS3 expression. CerS3 expression in human keratinocytes (HK) treated with PPARδ agonist (GW501516, 1 μmol/ml) or antagonist (GSK0660, 1 μmol/ml) as measured by qPCR. Data presented as mean±SEM (n=5). FIGS. 11D-11F: Upregulation of DNA methyl transferases in S-1-P treated HK. Data presented as mean±SEM (n=8). FIGS. 11G-11J: Induction of histone deacetylases in S-1P treated HK. Data presented as mean±SEM (n=8). FIGS. 11K-11N) Other histone deacetylases in S-1P treated HK. No significant changes (NS) were observed. Data presented as mean±SEM, (n=8). FIG. 11O: miR-106b expression in HK treated with S-1-P (5 μmol/ml). Data presented as mean±SEM (n=8).

FIGS. 12A-12C illustrate PPARδ drives ABCA12 expression. FIGS. 12A-12B) PPARδ drives ABCA12 expression in human keratinocytes (HK). HK treated (24 h) with PPARδ agonist (GW501516, 1 μmol/ml) or antagonist (GSK0660, 1 μmol/ml) as measured by qPCR. Data presented as mean±SEM (n=5). FIG. 12C: Downregulation of ABCA12 in PA LCM captured PA infected wound epithelium compared to PAΔcer qPCR; data presented as mean±SEM (n=5).

### DETAILED DESCRIPTION

### Definitions

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in a patent, application, or other publication that is herein incorporated by reference, the definition set forth in this section prevails over the definition incorporated herein by reference. In describing and claiming the invention, the following terminology will be used in accordance with the definitions set forth below.

The singular forms “a,” “an,” and “the” include plural referents unless the context clearly dictates otherwise. The terms “including,” “containing,” and “comprising” are used in their open, non-limiting sense.

The term “about” as used herein means greater or lesser than the value or range of values stated by 10 percent but is not intended to limit any value or range of values to only this broader definition. Each value or range of values preceded by the term “about” is also intended to encompass the embodiment of the stated absolute value or range of values.

As used herein, the term “purified” and like terms relate to the isolation of a molecule or compound in a form that is substantially free of contaminants normally associated with the molecule or compound in a native or natural environment. As used herein, the term “purified” does not require absolute purity; rather, it is intended as a relative definition. The term “purified polypeptide” is used herein to describe a polypeptide which has been separated from other compounds including, but not limited to nucleic acid molecules, lipids and carbohydrates.

The term “isolated” requires that the referenced material be removed from its original environment (e.g., the natural environment if it is naturally occurring). For example, a naturally-occurring polynucleotide present in a living animal is not isolated, but the same polynucleotide, separated from some or all of the coexisting materials in the natural system, is isolated.

As used herein the term “patient” without further designation is intended to encompass any warm blooded vertebrate domesticated animal (including for example, but not limited to livestock, horses, cats, dogs and other pets) and humans receiving therapeutic care with or without physician oversight.

The term “inhibit” refers to a decrease in an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

As used herein the term “ceramides” defines a family of waxy lipid molecules composed of sphingosine and a fatty acid and having the general structure of

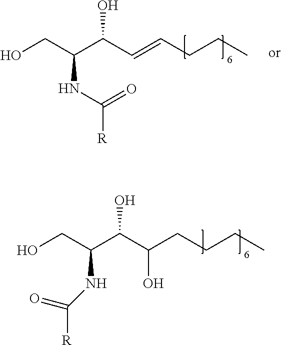

wherein R is a saturated or unsaturated alkyl chain of a fatty acid optionally wherein the fatty acid chain is extended by attachment of the group —O(CO)(CH₂)₇(CH═CH)CH₂(CH═CH)(CH₂)₄CH₃ to the free terminus of the fatty acid chain.

Ceramidases catalyze the degradation of ceramide to sphingosine and fatty acids.

As used herein a “ceramidase inhibitor” is any compound that exhibits inhibitory activity against a ceramidase such as a neutral/alkaline bacterial ceramidase. The bacterial ceramidase inhibitor may be obtained using bacterial ceramidase inhibitor-producing microorganisms or obtained through chemical synthesis. Ceramidastin is an exemplary bacterial ceramidase inhibitor described in U.S. Pat. No. 8,263,369, the disclosure of which is expressly incorporated herein. The structure of Ceramidastin is:

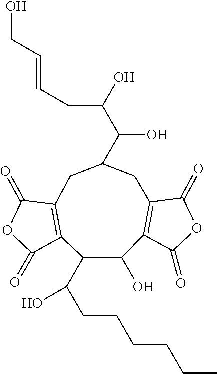 [SMILES: CCCCCCC(O)C1C2=C(CC(C(O)C(O)C/C=C/CO)CC3=C(C(=O)OC3=O)C1O)C(=O)OC2=O]

The term “controlled release” as used herein refers to a formulation in which the manner and profile of drug release from the formulation is controlled. This includes immediate as well as non-immediate release formulations, with non-immediate release formulations including, but not limited to, sustained release and delayed release formulations. The term “sustained release” (also referred to as “extended release”) is used herein in its conventional sense to refer to a drug formulation that provides for gradual release of a drug over an extended period of time, and that may result in substantially constant levels of a drug over an extended time period. The term “delayed release” is used herein in its conventional sense to refer to a drug formulation in which there is a time delay between administration of the formulation and the release of the drug therefrom. “Delayed release” may or may not involve gradual release of drug over an extended period of time, and thus may or may not be “sustained release.” The term “long-term” release, as used herein, means that the drug formulation is constructed and arranged to deliver therapeutic levels of the active ingredient for at least: 2 hours, 3 hours, 4 hours, hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 49 hours, 50 hours, 51 hours, 52 hours, 53 hours, 54 hours, 55 hours, 56 hours, 57 hours, 58 hours, 59 hours, 60 hours, 61 hours, 62 hours, 63 hours, 64 hours, 65 hours, 66 hours, 67 hours, 68 hours, 69 hours, 70 hours, 71 hours, 72 hours, 73 hours, 74 hours, 75 hours, 76 hours, 77 hours, 78 hours, 79 hours, 80 hours, 81 hours, 82 hours, 83 hours, 84 hours, 85 hours, 86 hours, 87 hours, 88 hours, 89 hours, 90 hours, 91 hours, 92 hours, 93 hours, 94 hours, 95 hours, 96 hours, 97 hours, 98 hours, 99 hours, 100 hours, 101 hours, 102 hours, 103 hours, 104 hours, 105 hours, 106 hours, 107 hours, 108 hours, 109 hours, 110 hours, 111 hours, 112 hours, 113 hours, 114 hours, 115 hours, 116 hours, 117 hours, 118 hours, 119 hours, or 120 hours.

“Excipient” refers to any inactive ingredient that is added to the composition and that is not intended to exert therapeutic effects at the intended dosage, although it may act to improve product delivery. Additional characteristics of excipients can be found in the Guidance for Industry Nonclinical Studies for the Safety Evaluation of Pharmaceutical Excipients issued by the US Food and Drug Administration Center for Drug Evaluation and Research (May, 2005), herein incorporated by reference.

“Pharmaceutically acceptable carrier” refers to any substantially non-toxic carrier useable for formulation and administration of the composition of the described invention in which the product of the described invention will remain stable and bioavailable. The pharmaceutically acceptable carrier must be of sufficiently high purity and of sufficiently low toxicity to render it suitable for administration to the subject being treated. It further should maintain the stability and bioavailability of an active agent. The pharmaceutically acceptable carrier can be liquid or solid and is selected, with the planned manner of administration in mind, to provide for the desired bulk, consistency, etc., when combined with an active agent and other components of a given composition.

“Reduced” or “to reduce” refers to a diminution, a decrease, an attenuation or abatement of the degree, intensity, extent, size, amount, density or number.

“Topical” refers to administration of a pharmaceutical composition at, or immediately beneath, the point of application. The terms “topically”, “topical administration” and “topically applying” are used interchangeably to refer to delivering a pharmaceutical composition onto one or more surfaces of a tissue or cell, including epithelial surfaces. The composition may be applied by pouring, dropping, or spraying, if a liquid; rubbing on, if an ointment, lotion, cream, gel, or the like; dusting, if a powder; spraying, if a liquid or aerosol composition; or by any other appropriate means. Topical administration generally provides a local rather than a systemic effect.

“Treat” or “treating” includes abrogating, substantially inhibiting, slowing or reversing the progression of a disease, condition or disorder, substantially ameliorating clinical or esthetical symptoms of a condition, substantially preventing the appearance of clinical or esthetical symptoms of a disease, condition, or disorder, and protecting from harmful or annoying symptoms. “Treat” or “treating” further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously symptomatic for the disorder(s).

As used herein an “effective” amount or a “therapeutically effective amount” of a drug refers to a nontoxic but sufficient amount of the drug to provide the desired effect. The amount that is “effective” will vary from subject to subject or even within a subject overtime, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

“Wound healing agent” refers to an agent that promotes an intricate process where the skin or other body tissue repairs itself after injury. In normal skin, the epidermis (surface layer) and dermis (deeper layer) form a protective barrier against the external environment. As such, the term “wound healing agent” refers to any substance that facilitates the wound healing process.

### EMBODIMENTS

The present disclosure is directed to the discovery that microbial pathogens must co-opt host skin lipids to generate complex biofilms. Accordingly, anti-biofilm strategies must not necessarily always target the microbe itself. Targeting host lipids that are utilized by microbial pathogens, particularly under conditions where the risk of infection of the host by a microbial pathogen is enhanced, could be an effective strategy to prevent or treat such infections. In one embodiment an anti-biofilm strategy comprises the administration of both an agent that prevents microbial utilization of host skin lipids and an antimicrobial agent. In one embodiment a method of treating or preventing a microbial pathogenic infection is provided wherein the method comprises administering an agent that interferes or inhibits the pathogenic ceramide degrading mechanism of the microbial pathogen.

In one embodiment, a method of treating damaged tissue that exhibits a loss in barrier function with associated water loss/moisturization problems is provided wherein the damaged tissue is contacted with an exogenous source of epidermal lipids, selected from the group consisting of phospholipids, sphingolipids, cholesterol, free fatty acids, and triglycerides. In one embodiment the damaged tissue is contacted with a composition comprising a ceramide. In one embodiment the tissue is damaged as a result of a current, or previous, infection by a pathogenic microbial pathogen.

In accordance with one embodiment a composition is provided comprising an inhibitor of microbial lipases and one or more epidermal lipids. In one embodiment the composition comprises a ceramidase inhibitor. In one embodiment the composition comprises a ceramidase inhibitor and one or more ceramides. Accordingly, in one embodiment the present disclosure provides a method to inhibit pathogenic ceramide degrading mechanism of microbial as well as replenishing host ceramide to rescue barrier function of the repaired skin enabling functional wound closure. The ceramidase inhibitor in one embodiment is a compound that interferes with the enzymatic activity of ceramidase. Such compounds may include Ceramidastin, or hydroxypropyl bispalmitamide MEA. In one embodiment the ceramidase inhibitor is an agent that interferes with the expression of ceramidase, including for example an interference RNA that binds to the nucleic acids encoding ceramidase.

In accordance with one embodiment a pharmaceutical composition formulated for topical administration is provided wherein the composition comprises a bacterial ceramidase inhibitor and a pharmaceutically acceptable carrier. In one embodiment the composition includes an amount of a bacterial ceramidase inhibitor that is greater than zero to about 10 wt. %, based on the total weight of the composition. For example, the total amount of ceramides may be from greater than zero to about 9 wt. %, greater than zero to about 8 wt. %, greater than zero to about 7 wt. %, greater than zero to about 6 wt. %, greater than zero to about 5 wt. %, greater than zero to about 4 wt. %, greater than zero to about 3 wt. %, greater than zero to about 2 wt. %, greater than zero to about 1 wt. %, based on the total weight of the composition. In one embodiment the composition further comprises one or more epidermal lipids selected from ceramides, phospholipids, sphingolipids, cholesterol, free fatty acids, and triglycerides.

In accordance with one embodiment the ceramidase inhibitor comprising compositions of the present disclosure further comprise one or more ceramides or derivative thereof. In one embodiment the ceramides of the composition have the general formula:

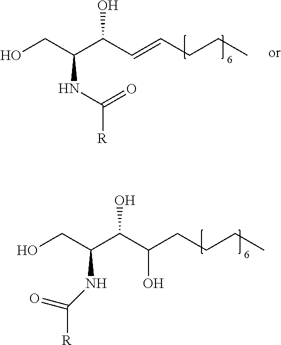

wherein R is a saturated or unsaturated aliphatic chain of carbon atoms selected from the range of 4 to 32, optionally wherein R is —(CH₂)₁₈₋₂₉(CH₃), —(CHOH)(CH₂)₁₈₋₂₉(CH₃) or —(CH₂)₁₈₋₃₀O(CO)(CH₂)₇(CH═CH)CH₂(CH═CH)(CH₂)₄CH₃. In one embodiment the ceramide is selected from the group consisting of ceramide 1, ceramide 2, ceramide 3, ceramide 3B, ceramide 4, ceramide 5, ceramide 1A, ceramide 6 II, ceramide AP, ceramide EOP, ceramide EOS, ceramide NP, ceramide NG, ceramide NS, ceramide AS, ceramide NS dilaurate, and a mixture thereof. In some instances, the ceramides include or may be chosen from ceramide-EOS, ceramide-NS, ceramide-NP, ceramide-EOH, ceramide-AS, ceramide-NH, ceramide-AP, ceramide-AH, ceramide-OS, ceramide-OH, (pseudo-)ceramides such as hydroxypropyl bispalmitamide MEA, cetyloxypropyl glyceryl methoxypropyl myristamide, N-(1-hexadecanoyl)-4-hydroxy-L-proline (1-hexadecyl) ester, and hydroxyethyl palmityl oxyhydroxypropyl palmitamide, and a mixture thereof. In some embodiments, the compositions may include ceramide EOP, optionally, in combination with one, two, or more other ceramides. For instance, in some instances, the compositions includes a combination of ceramides, for example, a combination of ceramide EOP, ceramide NP, Ceramide AP.

Ceramides and ceramide derivatives include, but are not limited to, derivatives of the SN-1 position including 1-chloro and 1-benzoyl ceramides, which would not be subject to phosphorylation at this position, as well as derivatives at the SN-2 position (amide linkage), such as a methylcarbamate group or a 2-0-ethyl substituent, which would not be subject to degradation by ceramidases. In addition, cell-permeable forms of these ceramides analogs can be utilized. Examples of these cell-permeable ceramides and/or derivatives contain 2-10 carbons and have short chain fatty acids at the SN-2 position (C6 ceramide). An example of C6-ceramide is N-Hexanoyl-D-erythro-Sphingosine. Ceramides may be isolated from natural sources or chemically synthesized. In one embodiment, the ceramide is already formulated in a commercially available cream, lotion, spray, gel, foam or ointment. Examples of commercially available compositions include: EpiCeram® produced by Promius Pharma, CERAVE® (New York, NY) or CERAMEDX® (Santa Barbara, CA). In one embodiment the composition includes an amount of one or more ceramides that is greater than zero to about 10 wt. %, based on the total weight of the composition. For example, the total amount of ceramides may be from greater than zero to about 9 wt. %, greater than zero to about 8 wt. %, greater than zero to about 7 wt. %, greater than zero to about 6 wt. %, greater than zero to about 5 wt. %, greater than zero to about 4 wt. %, greater than zero to about 3 wt. %, greater than zero to about 2 wt. %, greater than zero to about 1 wt. %, based on the total weight of the composition.

In one embodiment the compositions of the present disclosure include a sterol. Exemplary sterols include cholesterol, cholesteryl sulfate, cholesteryl acetate, cholesteryl stearate, cholesteryl isostearate, cholesteryl hydroxystearate, and phytosterol. The total amount of sterol in the composition may vary from about 0.1 to about 20 wt. % based on the total weight of the composition. For example, the total amount of cholesterol may be 0.1 wt. % or more, 0.2 wt. % or more, 0.3 wt. % or more, 0.4 wt. % or more, 0.5 wt. % or more, 0.6 wt. % or more, 0.7 wt. % or more, 0.8 wt. % or more, 0.9 wt. % or more, 1.0 wt. % or more and/or 20 wt. % or less, 15 wt. % or less, 13 wt. % or less, 11 wt. % or less, 10 wt. % or less, 9 wt. % or less, 8 wt. % or less, 7 wt. % or less, 6 wt. % or less, 5 wt. % or less, 4 wt. % or less, 3 wt. % or less, 2.5 wt. % or less, including ranges and sub-ranges therebetween, based on the total weight of the composition.

The compositions may include water, e.g., purified water. The total amount of water in the composition can vary, but is typically about 30 to about 95 wt. %, based on the total weight of the cleansing composition. In some instances, total amount of water is about 30 to about 90 wt. %, about 30 to about 85 wt. %, about 30 to about 80 wt. %, about 35 to about 90 wt. %, about 35 to about 85 wt. %, about 35 to about 80 wt. %, about 40 to about 90 wt. %, about 40 to about 85 wt. %, about 40 to about 80 wt. %, about 45 to about 90 wt. %, about 45 to about 85 wt. %, about 45 to about 80 wt. %, about 50 to about 90 wt. %, about 50 to about 85 wt. %, about 50 to about 80 wt. %, about 55 to about 90 wt. %, about 55 to about 85 wt. %, about 55 to about 80 wt. %, about 60 to about 90 wt. %, about 60 to about 85 wt. %, about 60 to about 80 wt. %, about 65 to about 90 wt. %, about 65 to about 85 wt. %, or about 65 to about 80 wt. %, based on the total weight of the composition.

In one embodiment the compositions further include fatty acid compounds such as unsaturated or saturated fatty acids or fatty acid derivatives. The unsaturated or saturated fatty acids include, but are not limited to, butyric acid, caproic acid, caprylic acid, capric acid, lauric acid, myristic acid, myristoleic acid, palmitic acid, palmitoleic acid, stearic acid, oleic acid, linoleic acid, linolenic acid, arachidic acid, gadoleic acid, pentadecanoic acid, margaric acid, margaroleic acid, behenic acid, dihomolinoleic acid, arachidonic acid and lignoceric acid. The fatty acid derivatives are defined to include fatty acid esters of the fatty alcohols, fatty acid esters of the fatty alcohol derivatives when such fatty alcohol derivatives have an esterifiable hydroxyl group, fatty acid esters of alcohols other than the fatty alcohols and the fatty alcohol derivatives, hydroxy-substituted fatty acids, and a mixture thereof. Nonlimiting examples of fatty acid derivatives include conjugated linoleic acid, ricinoleic acid, glycerol monostearate, 12-hydroxy stearic acid, ethyl stearate, cetyl stearate, cetyl palmitate, polyoxyethylene cetyl ether stearate, polyoxyethylene stearyl ether stearate, polyoxyethylene lauryl ether stearate, ethyleneglycol monostearate, polyoxyethylene monostearate, polyoxyethylene distearate, propyleneglycol monostearate, propyleneglycol distearate, trimethylolpropane distearate, sorbitan stearate, glyceryl stearate, polyglyceryl stearate, dimethyl sebacate, PEG-15 cocoate, PPG-15 stearate, glyceryl monostearate, glyceryl distearate, glyceryl tristearate, PEG-8 laurate, PPG-2 isostearate, PPG-9 laurate, and a mixture thereof.

The total amount of fatty compounds in the compositions may vary from, e.g., about 0.1 to about 25 wt. %, based on the total weight of the composition. For example, the total amount of fatty compounds may be from about 0.1 to about 25 wt. %, about 0.1 to about 20 wt. %, from about 0.1 to about 15 wt. %, about 0.1 to about 10 wt. %, about 0.1 to about 8 wt. %, about 0.1 to about 6 wt. %, from about 0.5 to about 25 wt. % about 0.5 to about 20 wt. %, about 0.5 to about 15 wt. %, about 0.5 to 20 about 10 wt. %, about 0.5 to about 8 wt. %, about 0.5 to 6 wt. %, from about 1 to about 25 wt. %, about 1 to about 20 wt. %, about 1 to about 15 wt. %, about 1 to about 10 wt. %, about 1 to about 8 wt. %, or about 1 to about 6 wt. %, from about 1.5 to about 25 wt. %, about 1.5 to about 20 wt. %, about 1.5 to about 15 wt. %, about 1.5 to about 10 wt. %, about 1.5 to about 8 wt. %, or about 1.5 to about 6 wt. %, from about 2 to about 25 wt. %, about 2 to about 20 wt. %, about 2 to about 15 wt. %, about 2 to about 10 wt. %, about 2 to about 8 wt. %, or about 2 to about 6 wt. %, from about 2.5 to about 25 wt. %, about 2.5 to about 20 wt. %, about 2.5 to about 15 wt. %, about 2.5 to about 10 wt. %, about 2.5 to about 8 wt. %, or about 2.5 to about 6 wt. %, from about 3 to about 25 wt. %, about 3 to about 20 wt. %, about 3 to about 15 wt. %, about 3 to about 10 wt. %, about 3 to about 8 wt. %, or about 3 to about 6 wt. %, from about 3.5 to about 25 wt. %, about 3.5 to about 20 wt. %, about 3.5 to about 15 wt. %, about 3.5 to about 10 wt. %, about 3.5 to about 8 wt. %, or about 3.5 to about 6 wt. %, including ranges and sub-ranges therebetween, based on the total weight of the composition. Additionally or alternatively, the total amount of fatty compounds may be from 0.1 to 25 wt. %, 0.1 to 20 wt. %, from 0.1 to 15 wt. %, 0.1 to 10 wt. %, 0.1 to 8 wt. %, 0.1 to 6 wt. %, from 0.1 to 25 wt. %, 0.5 to 20 wt. %, 0.5 to 15 wt. %, 0.5 to 10 wt. %, 0.5 to 8 wt. %, 0.5 to 6 wt. %, from 1 to 25 wt. %, 1 to 20 wt. %, 1 to 15 wt. %, 1 to 10 wt. %, 1 to 8 wt. %, or 1 to 6 wt. %, from 1.5 to 25 wt. %, 1.5 to 20 wt. %, 1.5 to 15 wt. %, 1.5 to 10 wt. %, 1.5 to 8 wt. %, or 1.5 to 6 wt. %, from 2 to 25 wt. %, 2 to 20 wt. %, 2 to 15 wt. %, 2 to 10 wt. %, 2 to 8 wt. %, or 2 to 6 wt. %, from 2.5 to 25 wt. %, 2.5 to 20 wt. %, 2.5 to 15 wt. %, 2.5 to 10 wt. %, 2.5 to 8 wt. %, or 2.5 to 6 wt. %, from 3 to 25 wt. %, 3 to 20 wt. %, 3 to 15 wt. %, 3 to 10 wt. %, 3 to 8 wt. %, or 3 to 6 wt. %, from 3.5 to 25 wt. %, 3.5 to 20 wt. %, 3.5 to 15 wt. %, 3.5 to 10 wt. %, 3.5 to 8 wt. %, or 3.5 to 6 wt. %, including ranges and sub-ranges therebetween, based on the total weight of the composition.

Non-limiting examples of fatty compounds of the composition include or may be chosen from oils, mineral oil, fatty alcohols, fatty acids, fatty alcohol derivatives, fatty acid derivatives (e.g., alkoxylated fatty acids or polyethylene glycol esters of fatty acids or propylene glycol esters of fatty acids or butylene glycol esters of fatty acids or esters of neopentyl glycol and fatty acids or polyglycerol/glycerol esters of fatty acids or glycol diesters or diesters of ethylene glycol and fatty acids or esters of fatty acids and fatty alcohols, esters of short chain alcohols and fatty acids), glyceryl esters (glycerol esters), alkyl ethers of fatty alcohols, fatty acid esters of alkyl ethers of fatty alcohols, fatty acid esters of alkoxylated fatty alcohols, fatty acid esters of alkyl ethers of alkoxylated fatty alcohols, esters of fatty alcohols, hydroxy-substituted fatty acids, waxes, triglyceride compounds, lanolin, and a mixture thereof. In some instances, the one or more fatty compound may comprise or be chosen from fatty alcohols, fatty acids, esters of fatty acids, and/or esters of fatty alcohols (e.g., cetyl palmitate, cetyl stearate, myristyl myristate, myristyl stearate, cetyl myristate, and stearyl stearate (a mixture of which is referred to as “cetyl esters”)). Additionally or alternatively, the one or more fatty compounds may include or be chosen from hydrocarbons, fatty alcohols, fatty alcohol derivatives, fatty acids, fatty acid derivatives, fatty esters, fatty ethers, oils, waxes, etc. In one instance, the one or more fatty compounds is a hydrocarbon that is linear, branched, and/or cyclical, such as cyclic C6-C16 alkanes, hexane, undecane, dodecane, tridecane, and isoparaffins, for instance isohexadecane, isododecane and isodecane. Additionally, the linear or branched hydrocarbons may be composed only of carbon and hydrogen atoms of mineral, plant, animal or synthetic origin with more than 16 carbon atoms, such as volatile or non-volatile liquid paraffins, petroleum jelly, liquid petroleum jelly, petrolatum polydecenes, hydrogenated polyisobutene, and squalane.

In one embodiment the composition includes one or more polyols. The one or more polyols may be chosen from polyols having from 2 to 15 carbon atoms and at least two hydroxyl groups. Exemplary polyols that may be used in the composition include and/or may be chosen from alkanediols such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; sorbitol; sorbitan; triacetin; and a mixture thereof.

The one or more polyols may be glycols or glycol ethers such as, e.g., monomethyl, monoethyl and monobutyl ethers of ethylene glycol, propylene glycol or ethers thereof such as, e.g., monomethyl ether of propylene glycol, butylene glycol, hexylene glycol, dipropylene glycol as well as alkyl ethers of diethylene glycol, e.g., monoethyl ether or monobutyl ether of diethylene glycol. In one instance, the one or more polyols include or are chosen from ethylene glycol, propylene glycol, butylene glycol, hexylene glycol, pentylene glycol, 1,3-propanediol, diethylene glycol, dipropylene glycol, caprylyl glycol, glycerin, and a mixture thereof. In another instance, the composition includes or is chosen from caprylyl glycol, glycerin, and a mixture thereof.

In some embodiments, the composition has an amount of propylene glycol and/or ethoxydiglycol that is less than about 10 wt. %, preferably less than about 9 wt. %, preferably less than about 8 wt. %, preferably less than about 7 wt. %, preferably less than about 6 wt. %, preferably less than about 5 wt. %, preferably less than about 4 wt. %, preferably less than about 3 wt. %, preferably less than about 2 wt. %, or preferably less than about 1 wt. %, based on the total weight of the composition. The composition may, alternatively, have an amount of propylene glycol and/or ethoxydiglycol that is less than 10 wt. %, preferably less than 9 wt. %, preferably less than 8 wt. %, preferably less than 7 wt. %, preferably less than 6 wt. %, preferably less than about 5 wt. %, preferably less than 4 wt. %, preferably less than 3 wt. %, preferably less than 2 wt. %, or preferably less than 1 wt. %, based on the total weight of the composition. In one instance, the one or more polyols does not include propylene glycol and/or ethoxydiglycol, such that the composition is free or essentially free of propylene glycol and/or ethoxydiglycol.

The total amount of polyols in the compositions may vary from, e.g., about 0.1 to about 25 wt. %, based on the total weight of the composition. For example, the total amount of polyols may be from about 0.1 to about 25 wt. %, about 0.1 to about 20 wt. %, from about 0.1 to about 15 wt. %, about 0.1 to about 10 wt. %, about 0.1 to about 8 wt. %, about 0.1 to about 6 wt. %, from about 0.5 to about 25 wt. % about 0.5 to about 20 wt. %, about 0.5 to about 15 wt. %, about 0.5 to about 10 wt. %, about 0.5 to about 8 wt. %, about 0.5 to 6 wt. %, from about 1 to about 25 wt. %, about 1 to about 20 wt. %, about 1 to about 15 wt. %, about 1 to about 10 wt. %, about 1 to about 8 wt. %, or about 1 to about 6 wt. %, from about 1.5 to about 25 wt. %, about 1.5 to about 20 wt. %, about 1.5 to about 15 wt. %, about 1.5 to about 10 wt. %, about 1.5 to about 8 wt. %, or about 1.5 to about 6 wt. %, from about 2 to about 25 wt. %, about 2 to about 20 wt. %, about 2 to about 15 wt. %, about 2 to about 10 wt. %, about 2 to about 8 wt. %, or about 2 to about 6 wt. %, from about 2.5 to about 25 wt. %, about 2.5 to about 20 wt. %, about 2.5 to about 15 wt. %, about 2.5 to about 10 wt. %, about 2.5 to about 8 wt. %, or about 2.5 to about 6 wt. %, from about 3 to about 25 wt. %, about 3 to about 20 wt. %, about 3 to about 15 wt. %, about 3 to about 10 wt. %, about 3 to about 8 wt. %, or about 3 to about 6 wt. %, from about 3.5 to about 25 wt. %, about 3.5 to about 20 wt. %, about 3.5 to about 15 wt. %, about 3.5 to about 10 wt. %, about 3.5 to about 8 wt. %, or about 3.5 to about 6 wt. %, including ranges and sub-ranges therebetween, based on the total weight of the composition. Additionally or alternatively, the total amount of polyols may be from 0.1 to 25 wt. %, 0.1 to 20 wt. %, from 0.1 to 15 wt. %, 0.1 to 10 wt. %, 0.1 to 8 wt. %, 0.1 to 6 wt. %, from 0.1 to 25 wt. %, 0.5 to 20 wt. %, 0.5 to 15 wt. %, 0.5 to 10 wt. %, 0.5 to 8 wt. %, 0.5 to 6 wt. %, from 1 to 25 wt. %, 1 to 20 wt. %, 1 to 15 wt. %, 1 to 10 wt. %, 1 to 8 wt. %, or 1 to 6 wt. %, from 1.5 to 25 wt. %, 1.5 to 20 wt. %, 1.5 to 15 wt. %, 1.5 to 10 wt. %, 1.5 to 8 wt. %, or 1.5 to 6 wt. %, from 2 to 25 wt. %, 2 to 20 wt. %, 2 to 15 wt. %, 2 to 10 wt. %, 2 to 8 wt. %, or 2 to 6 wt. %, from 2.5 to 25 wt. %, 2.5 to 20 wt. %, 2.5 to 15 wt. %, 2.5 to 10 wt. %, 2.5 to 8 wt. %, or 2.5 to 6 wt. %, from 3 to 25 wt. %, 3 to 20 wt. %, 3 to 15 wt. %, 3 to 10 wt. %, 3 to 8 wt. %, or 3 to 6 wt. %, from 3.5 to 25 wt. %, 3.5 to 20 wt. %, 3.5 to 15 wt. %, 3.5 to 10 wt. %, 3.5 to 8 wt. %, or 3.5 to 6 wt. %, including ranges and sub-ranges therebetween, based on the total weight of the composition.

The compositions may include one or more emulsifiers. For example, the emulsifier may be an amphoteric, anionic, cationic or nonionic emulsifier, used alone or as a mixture, and optionally with a co-emulsifier. The emulsifiers are chosen in an appropriate manner according to the emulsion to be obtained.

In some cases, the nonionic surfactant may be chosen from esters of polyols with fatty acids with a saturated or unsaturated chain containing for example from 8 to 24 carbon atoms, and alkoxylated derivatives thereof; polyethylene glycol esters of a C8-C24; sorbitol esters of a C8-C24; sugar (sucrose, glucose, alkylglycose) esters of a C8-C24, preferably C12-C22, fatty acid or acids and alkoxylated derivatives thereof; ethers of sugar and a C8-C24, preferably C12-C22, fatty alcohol or alcohols; and mixtures thereof. In one instance, the nonionic surfactant is an ethoxylated fatty ester chosen from adducts of ethylene oxide with esters of lauric acid, palmitic acid, stearic acid or behenic acid, and mixtures thereof. Examples of ethoxylated fatty esters that may be suitable include those containing from 9 to 100 oxyethylene groups, such as PEG-9 to PEG-50 laurate (as the CTFA names: PEG-9 laurate to PEG-50 laurate); PEG-9 to PEG-50 palmitate (as the CTFA names: PEG-9 palmitate to PEG-50 palmitate); PEG-9 to PEG-50 stearate (as the CTFA names: PEG-9 stearate to PEG-50 stearate); PEG-9 to PEG-50 palmitostearate; PEG-9 to PEG-50 behenate (as the CTFA names: PEG-9 behenate to PEG-50 behenate); polyethylene glycol 100 EO monostearate (CTFA name: PEG-100 stearate); and mixtures thereof.

In some instances, the composition may include an emulsifier such as dimers surfactants which may have two surfactant moieties identical or different, and constituted by a hydrophilic head group and a lipophilic group linked to each other through the head groups, thanks to a spacer. For example, the one or more emulsifiers may include or be chosen from those sold by Sasol company under the name CERALUTIOM, for example, CERALUTION H: Behenyl Alcohol, Glyceryl Stearate, Glyceryl Stearate Citrate et Sodium Dicocoyl ethylenediamine PEG-15 Sulfate, CERALUTION F: Sodium Lauroyl Lactylate et Sodium Dicocoyl ethylenediamine PEG-15 Sulfate, CERALUTION C: Aqua, Capric/Caprylic triglyceride, Ceteareth-25, Sodium Dicocoyl ethylenediamine PEG-15 Sulfate, Sodium Lauroyl Lactylate, Behenyl Alcohol, Glyceryl Stearate, Glyceryl Stearate Citrate, Gum Arabic, Xanthan Gum, Phenoxyethanol, Methylparaben, Ethylparaben, Butylparaben, Isobutylparaben. In one embodiment, the emulsifier of the composition consists of sodium lauroyl lactylate or consists essentially of sodium lauroyl lactylate. In another embodiment, the emulsifier(s) of the composition includes sodium lauroyl lactylate with one or more additional emulsifiers, such as a nonionic emulsifier or an anionic emulsifier.

The total amount of emulsifiers in the compositions may vary from, e.g., about 0.001 to about 25 wt. %, based on the total weight of the composition. For example, the total amount of fatty compounds may be from about 0.001 to about 25 wt. %, about 0.001 to about 20 wt. %, from about 0.001 to about 15 wt. %, about 0.001 to about 10 wt. %, about 0.001 to about 8 wt. %, about 0.001 to about 6 wt. %, from about 0.005 to about 25 wt. % about 0.005 to about 20 wt. %, about 0.005 to about 15 wt. %, about 0.005 to about 10 wt. %, about 0.005 to about 8 wt. %, about 0.005 to 6 wt. %, from about 0.01 to about 25 wt. %, about 0.01 to about 20 wt. %, about 0.01 to about 15 wt. %, about 0.01 to about 10 wt. %, about 0.01 to about 8 wt. %, or about 0.01 to about 6 wt. %, from about 0.05 to about 25 wt. %, about 0.05 to about 20 wt. %, about 0.05 to about 15 wt. %, about 0.05 to about 10 wt. %, about 0.05 to about 8 wt. %, or about 0.05 to about 6 wt. % including ranges and sub-ranges therebetween, based on the total weight of the composition. In one instance, the total amount of emulsifiers in the composition are typically in an amount from 0.5, 0.6, 0.7, 0.8, 0.9 or 1.0 wt. % to 5, 6, 7, 8, 9, or 10 wt. %.

The composition may be formulated to have a lower amount of emulsifier(s) than typical commercial products. For example, the composition may have a total amount of emulsifiers ranging from about 0.001 to about 6 wt. %, about 0.001 to about 5 wt. %, from about 0.001 to about 4 wt. %, about 0.001 to about 3 wt. %, about 0.001 to about 2 wt. %, about 0.001 to about 1 wt. %, from about 0.005 to 6 wt. %, about 0.005 to about 5 wt. % about 0.005 to about 4 wt. %, about 0.005 to about 3 wt. %, about 0.005 to about 2 wt. %, about 0.005 to about 1 wt. %, from about 0.01 to about 6 wt. %, about 0.01 to about 5 wt. %, about 0.01 to about 4 wt. %, about 0.01 to about 3 wt. %, about 0.01 to about 2 wt. %, or about 0.01 to about 1 wt. %, from about 0.05 to about 6 wt. %, about 0.05 to about 5 wt. %, about 0.05 to about 4 wt. %, about 0.05 to about 3 wt. %, about 0.05 to about 2 wt. %, or about 0.05 to about 1 wt. % including ranges and sub-ranges therebetween, based on the total weight of the composition. Additionally or alternatively, the composition may have a total amount of emulsifiers ranging from 0.001 to 6 wt. %, 0.001 to 5 wt. %, from 0.001 to 4 wt. %, 0.001 to 3 wt. %, 0.001 to 2 wt. %, 0.001 to 1 wt. %, from 0.005 to 6 wt. %, 0.005 to 5 wt. % 0.005 to 4 wt. %, 0.005 to 3 wt. %, 0.005 to 2 wt. %, 0.005 to 1 wt. %, from 0.01 to 6 wt. %, 0.01 to 5 wt. %, 0.01 to 4 wt. %, 0.01 to 3 wt. %, 0.01 to 2 wt. %, or 0.01 to 1 wt. %, from 0.05 to 6 wt. %, 0.05 to 5 wt. %, 0.05 to 4 wt. %, 0.05 to 3 wt. %, 0.05 to 2 wt. %, or 0.05 to 1 wt. % including ranges and sub-ranges therebetween, based on the total weight of the composition.

The compositions described herein may comprise one or more silicone oils. Non-limiting examples of silicone oils include dimethicone, cyclomethicone, polysilicone-11, phenyl trimethicone, trimethylsilylamodimethicone, and stearoxytrimethylsilane. In some cases, the composition includes dimethicone, and optionally additional oils, including additional silicone oils. Typically, the one or more silicone oils is a non-volatile silicon oil. In some embodiments, the silicone oil is polydimethylsiloxanes (PDMSs), polydimethylsiloxanes comprising alkyl or alkoxy groups which are pendent and/or at the end of the silicone chain, which groups each contain from 2 to 24 carbon atoms, or phenyl silicones, such as phenyl trimethicones, phenyl dimethicones, phenyl(trimethylsiloxy)diphenylsiloxanes, diphenyl dimethicones, diphenyl(methyldiphenyl)trisiloxanes or (2-phenylethyl)trimethylsiloxysilicates.

The amount of the one or more silicone oils in the composition may vary from, e.g., about 0.1 to about 25 wt. %, based on the total weight of the composition. For example, the total amount of silicone oils may range from about 0.1 to about 25 wt. %, about 0.1 to about 20 wt. %, from about 0.1 to about 15 wt. %, about 0.1 to about 10 wt. %, about 0.1 to about 8 wt. %, about 0.1 to about 6 wt. %, from about 0.5 to about 25 wt. % about 0.5 to about 20 wt. %, about 0.5 to about 15 wt. %, about 0.5 to about 10 wt. %, about 0.5 to about 8 wt. %, about 0.5 to 6 wt. %, or about 0.5 to 5 wt. %, from about 1 to about 25 wt. %, about 1 to about 20 wt. %, about 1 to about 15 wt. %, about 1 to about 10 wt. %, about 1 to about 8 wt. %, about 1 to about 6 wt. %, or about 1 to 5 wt. %, from about 1.5 to about 25 wt. %, about 1.5 to about 20 wt. %, about 1.5 to about 15 wt. %, about 1.5 to about 10 wt. %, about 1.5 to about 8 wt. %, about 1.5 to about 6 wt. %, or about 1.5 to 5 wt. %, from about 2 to about 25 wt. %, about 2 to about 20 wt. %, about 2 to about 15 wt. %, about 2 to about 10 wt. %, about 2 to about 8 wt. %, about 2 to about 6 wt. %, or about 2 to 5 wt. %, from about 2.5 to about 25 wt. %, about 2.5 to about 20 wt. %, about 2.5 to about 15 wt. %, about 2.5 to about 10 wt. %, about 2.5 to about 8 wt. %, about 2.5 to about 6 wt. %, or about 2.5 to 5 wt. %, from about 3 to about 25 wt. %, about 3 to about 20 wt. %, about 3 to about 15 wt. %, about 3 to about 10 wt. %, about 3 to about 8 wt. %, about 3 to about 6 wt. %, or about 3 to 5 wt. %, including ranges and sub-ranges therebetween, based on the total weight of the composition.

Additionally or alternatively, the total amount of silicone oils may be from 0.1 to 25 wt. %, 0.1 to 20 wt. %, from 0.1 to 15 wt. %, 0.1 to 10 wt. %, 0.1 to 8 wt. %, 0.1 to 6 wt. %, from 0.5 to 25 wt. %, 0.5 to 20 wt. %, 0.5 to 15 wt. %, 0.5 to 10 wt. %, 0.5 to 8 wt. %, 0.5 to 6 wt. %, or 0.5 to 5 wt. %, from 1 to 25 wt. %, 1 to 20 wt. %, 1 to 15 wt. %, 1 to 10 wt. %, 1 to 8 wt. %, 1 to 6 wt. %, or 1 to 5 wt. %, from 1.5 to 25 wt. %, 1.5 to 20 wt. %, 1.5 to 15 wt. %, 1.5 to 10 wt. %, 1.5 to 8 wt. %, 1.5 to 6 wt. %, or 1.5 to 5 wt. %, from 2 to 25 wt. %, 2 to 20 wt. %, 2 to 15 wt. %, 2 to 10 wt. %, 2 to 8 wt. %, 2 to 6 wt. %, or 2 to 5 wt. %, from 2.5 to 25 wt. %, 2.5 to 20 wt. %, 2.5 to 15 wt. %, 2.5 to 10 wt. %, 2.5 to 8 wt. %, 2.5 to 6 wt. %, or 2.5 to 5 wt. %, from 3 to 25 wt. %, 3 to 20 wt. %, 3 to 15 wt. %, 3 to 10 wt. %, 3 to 8 wt. %, 3 to 6 wt. %, or 3 to 5 wt. %, including ranges and sub-ranges therebetween, based on the total weight of the composition.

One or more preservatives may be included in the compositions described herein. Suitable preservatives may include, but are not limited to, glycerin containing compounds (e.g., glycerin or ethylhexylglycerin or phenoxyethanol), benzyl alcohol, parabens (methylparaben, ethylparaben, propylparaben, butylparaben, isobutylparaben, etc.), sodium benzoate, ethylenediamine-tetraacetic acid (EDTA), disodium EDTA, potassium sorbate, and/or grapefruit seed extract, or combinations thereof. More than one preservative may be included in the composition. Other preservatives are known to those skilled in the art and include salicylic acid, DMDM Hydantoin, Formaldahyde, Chlorphenism, Triclosan, Imidazolidinyl Urea, Diazolidinyl Urea, Sorbic Acid, Methylisothiazolinone, Sodium Dehydroacetate, Dehydroacetic Acid, Quaternium-15, Stearalkonium Chloride, Zinc Pyrithione, Sodium Metabisulfite, 2-Bromo-2-Nitropropane, Chlorhexidine Digluconate, Polyaminopropyl biguanide, Benzalkonium Chloride, Sodium Sulfite, Sodium Salicylate, Citric Acid, Neem Oil, Essential Oils (various), Lactic Acid, and Vitamin E (tocopherol). In one instance, the composition has a plurality of preservatives including or chosen from disodium EDTA, phenoxyethanol, ethylhexylglycerin, tocopheryl acetate, and/or a mixture thereof.

The preservative is optionally included in an amount ranging from about 0.01 wt. % to about 5 wt. %, about 0.15% to about 1 wt. %, or about 1 wt. % to about 3 wt. %, based on the total weight of the composition.

The composition may include one or more pH adjusters to increase or decrease the overall pH of the composition. For example, one or more acids may be included to decrease the pH of the composition. Examples of suitable acids for decreasing the pH of the composition include, but are not limited to, citric acid, acetic acid, and the like. The composition may include one or more bases, such as sodium hydroxide, potassium hydroxide and the like, to decrease the pH of the composition. Additional or alternative acids and bases that are suitable for adjusting the pH of the composition are readily known to one of ordinary skill in the art.

The composition may, desirably, have a pH of pH of about 4 to about 7, preferably about 4.5 to about 6.5 or about 5.5 to about 6.5. Additionally or alternatively, the pH of the composition may range from 4 to 7, preferably from 4.5 to 6.5, or preferably from 5.5 to 6.5. In one instance, the pH of the composition is 6 or about 6.

The amount of the pH adjuster in the composition may be based on the desired pH of the final composition and/or product. For example, the total amount of the pH adjuster may range from about 0.05 to about 20 wt. %, based on the total weight of the composition. In some instances, the total amount of pH adjuster is from about 0.05 to about 15 wt. %, about 0.5 to about 10 wt. %, about 1 to about 5 wt. %, about 1.5 to about 4 wt. %, or about 2.0 to about 3 wt. %, including ranges and sub-ranges therebetween, based on the total weight of the composition. Additionally or alternatively, the compositions may include an amount of pH adjuster ranging from 0.05 to 15 wt. %, 0.5 to 10 wt. %, 1 to 5 wt. %, 1.5 to 4 wt. %, or 2.0 to 3 wt. %, including ranges and sub-ranges therebetween, based on the total weight of the composition.

The compositions may include one or more thickening agents. The amount of thickening agents may depend on the other components in composition and desired viscosity for the composition. For example, the composition may include an amount of thickening agents such that the viscosity of the composition is about 1,000 cP to about 100,000 cP, about 5,000 cP to about 50,000 cP, about 10,000 to about 50,000 cP, or about 15,000 cP to about 45,000 cP at a temperature of 25° C. using a Brookfield rheometer with a spindle number 5 at 20 revolutions per minute (RPM). Additionally or alternatively, the viscosity of the composition may be 1,000 cP to 100,000 cP, 5,000 cP to 50,000 cP, 10,000 to 50,000 cP, or 15,000 cP to 45,000 cP at a temperature of 25.degree. C. using a Brookfield rheometer with a spindle number 5 at 20 RPM.

The thickening agents may be in an amount of about 0.1 to about 20 wt. %, about 0.1 to about 10 wt. %, about 0.1 to about 9 wt. %, about 0.2 to about 9 wt. %, about 0.3 to about 9 wt. %, about 0.4 to about 8 wt. %, about 0.5 to about 5 wt. %, about 1 to about 20 wt. %, about 1 to about 5 wt. %, or about 1 to about 3 wt. %, including ranges and sub-ranges therebetween, based on the total weight of the composition. Additionally or alternatively, the thickening agents may be in an amount of 0.1 to 20 wt. %, 0.1 to 10 wt. %, 0.1 to 9 wt. %, 0.2 to 9 wt. %, 0.3 to 9 wt. %, 0.4 to 8 wt. %, 0.5 to 5 wt. %, 1 to 20 wt. %, 1 to 5 wt. %, or 1 to 3 wt. %, including ranges and sub-ranges therebetween, based on the total weight of the composition. Further, the amount of thickening agent(s) may be from 0.1, 0.2, 0.3, 0.4, 0.5, 0.6, 0.7, 0.8, 0.9, 1.0, or 1.5 wt. % to 2, 3, 4, 5, 6, 7, 8, 9, 10, 15, or 20 wt. %, including ranges and sub-ranges therebetween, based on the total weight of the composition.

The one or more thickening agent may be xanthan gum, guar gum, biosaccharide gum, cellulose, acacia Seneca gum, sclerotium gum, agarose, pechtin, gellan gum, hyaluronic acid. Additionally, the one or more thickening agent may include polymeric thickeners chosen from ammonium polyacryloyldimethyl taurate, ammonium acryloyldimethyltaurate/VP copolymer, sodium polyacrylate, acrylates copolymers, polyacrylamide, carbomer, and acrylates/C₁₀₋₃₀ alkyl acrylate crosspolymer. In one instance, the composition includes ammonium polyacryloyldimethyl taurate and/or sodium polyacrylate. In another instance, composition includes at least one or is chosen from ammonium polyacryloyldimethyl taurate, xanthan gum, carbomer, and a mixture thereof.

The compositions of the described invention may contain a microencapsulation component that is effective for keeping the active agent concentrated locally in the skin. According to some embodiments, the microencapsulation component comprises a liposome. According to some embodiments, the microencapsulation component comprises a polymer. According to some embodiments, the microencapsulation component comprises a complex of a liposome and a polymer or a polymersome. Other examples of microencapsulatiom components include, without limitation, micelles, reverse micelles, emulsions, microemulsions, etc.

Liposomes are generally known as sub-micron spherical vesicles comprised of phospholipids and cholesterol that form a hydrophobic bilayer surrounding an aqueous core. These structures have been used with a wide variety of therapeutic agents and allow for a drug to be entrapped within the liposome based in part upon its own hydrophobic (e.g. bilayer entrapment) or hydrophilic properties (e.g. entrapment in the aqueous compartment). Liposomes are generally used for controlled release and for drug targeting of lipid-capsulated compounds (Betageri et al, Liposome Drug Delivery Systems, Technomic Publishing Co., Inc., Lancaster, Pa., 1993).

Typically, encapsulating a drug, an active therapeutic agent or a pharmaceutical composition, such as in a liposome can alter the pattern of bio-distribution and the pharmacokinetics for the drugs. In certain cases, liposomal encapsulation has been found to lower drug toxicity. For example, long circulating liposomal formulations can avoid uptake by organs of the mononuclear phagocyte system, primarily in the liver and spleen. According to some embodiments, such long-circulating liposomes may include a surface coat of flexible water soluble polymer chains that act to prevent interaction between the liposome and plasma components that play a role in liposome uptake. According to some embodiments, such liposomes can be made of saturated, long-chain phospholipids and cholesterol, without this coating.

Exemplary liposomes may comprise a lipid layer comprising liposome forming lipids. The lipid may include at least one phosphatidyl choline which provides the primary packing/entrapment/structural element of the liposome. The phosphatidyl choline comprises mainly C16 or longer fatty-acid chains. Chain length provides for both liposomal structure, integrity, and stability. Optionally, one of the fatty-acid chains may have at least one double bond. As used herein, the term “phosphatidyl choline” includes, without limitation, soy PC, egg PC dielaidoyl phosphatidyl choline (DEPC), lecithin, dioleoyl phosphatidyl choline (DOPC), distearoyl phosphatidyl choline (DSPC), hydrogenated soybean phosphatidyl choline (HSPC), dipalmitoyl phosphatidyl choline (DPPC), 1-palmitoyl-2-oleo phosphatidyl choline (POPC), dibehenoyl phosphatidyl choline 30 (DBPC), and dimyristoyl phosphatidyl choline (DMPC).

Certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination. All combinations of the embodiments pertaining to the chemical groups represented by the variables are specifically embraced by the present disclosure and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present disclosure and are disclosed herein just as if each and every such sub-combination of chemical groups was individually and explicitly disclosed herein.

### Example 1

Ceramidastin Prevents Ceramide Loss by Pseudomonas aeruginosa Infection

### Methods

### Animals

Animals were housed under a 12-h light-dark cycle with food and water ad libitum.

### Bacterial Strains.

Pseudomonas aeruginosa wild type strain (PAwt), Pseudomonas aeruginosa ceramidase mutant (PAΔcer) were grown on Luria Agar (LA) plates or Luria broth with low sodium chloride (LBNS) at 37° C.

### Porcine Full Thickness Burn and Biofilm Wound Model

Domestic Yorkshire female pigs were wounded and infected to establish chronic wound biofilm model. Eight full thickness burn wounds (2×2 inch) were made on the dorsum of pigs. On day 3 post-burn, wounds were inoculated with either PAwt, or PAΔcer strains (CFU105/ml) in conjunction with Acinetobacter baumannii 19606 (CFU106/ml), another Gram-negative bacteria known to be abundant in chronic wounds. Control wounds were treated by mock inoculation with 250 μl of sterile PBS. Control wounds were not subjected to induced infection and were allowed to be colonized by skin microflora. These wounds are referred to as spontaneously infected (SI). Wounds were followed up to 56 days post inoculation. In chronic polymicrobial porcine wound biofilm infection comprising of Pseudomonas aeruginosa (PA), Acinetobacter baumannii (ACIN) and skin microflora, PA presents itself as the dominant species. Two sets of pigs were used trans-epidermal water loss (TEWL) pigs and biopsy pigs. TEWL pigs were only used for TEWL data collection on day 0, day 7, day 14 and day 56 post inoculation. Biopsy pigs were used only for biopsy collection on day 7, 14, 35 and 56 post-bacterial inoculation. The pigs were euthanized at day 56 post inoculation. Biopsies were collected using a 6 mm sterile disposable punch biopsy tool. Each independent porcine wound is considered as one “n” for the porcine wound experiments. Table 1 includes the total number of pigs used.

TABLE 1

| type | infection | pigs |
|---|---|---|
| TEWL pigs | SI | 3 |
| | PAwt | 6 |
| | PAΔcer | 5 |
| biopsy pigs | SI | 8 |
| | PAwt | 7 |
| | PAΔcer | 6 |
| total pigs | | 35 |

### Scanning Electron Microscope Imaging.

The samples were collected in glutaraldehyde fixation buffer, dehydrated with graded ethanol, and treated with hexamethyldisilazane (HMDS, Ted Pella Inc.) and left overnight for drying. Before scanning, samples were mounted and coated with gold. Imaging of the samples will be done by using a FEI™ NOVA nanoSEM scanning electron microscope (FEI™, Hillsboro, OR) equipped with a field-emission gun electron source.

### Trans-Epidermal Water Loss (TEWL) Measurement.

DermaLab Combo™ (cyberDERM inc., Broomall, PA) was used to measure the trans-epidermal water loss from the wounds. TEWL was measured in g (m2)-1 h-1. Dermalab Combo consists of a main measuring unit with a computer and a probe. The TEWL probe has two hygro sensors located close to each other in a perpendicular orientation, TEWL is determined from the humidity gradient between the sensors. The probe is placed on the porcine skin surface. The evaporated water released from the skin is detected by the sensors in the probe and measured to provide the TEWL value.

### Bacterial Ceramidase Activity Assay.

Pseudomonas ceramidase activity assay was adapted from Ohnishi. PAwt and PAΔcer strains were grown overnight in Luria broth with low sodium chloride (LBNS) at 37° C. PAwt and PAΔcer conditioned media was obtained from the overnight grown culture by filtering using 0.2 μm filters. The media (500 μl) was incubated with 15 mmol of C12-NBD-Ceramide for 16 h. The reaction was stopped by heating at 85° for 10 mins. The solution was evaporated using SpeedVac (Thermo Electron Corporation, Savant DNA120 SpeedVac Concentrator) and reconstituted in 50 μl of chloroform/methanol (2:1 v/v) followed by bath sonication for 10 mins. The solution is centrifuged at 1,500×g for 5 mins and the supernatant was used for and applied to a thin-layer chromatography (TLC) plate that was developed with chloroform/methanol/ammonia (90:15:1 v/v). The NBD-dodecanoic acid released by the action of the enzyme and the remaining NBD-ceramide were separated by TLC and imaged using aZure GelDoc (aZure biosystems c600). Image J (NIH) software was used for quantification of bands by densitometry.

Human Subjects and Fluid Collection from Chronic Wounds.

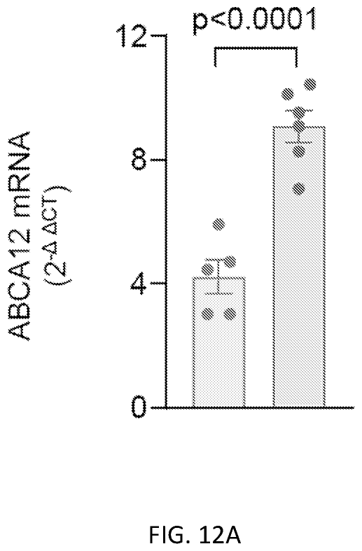

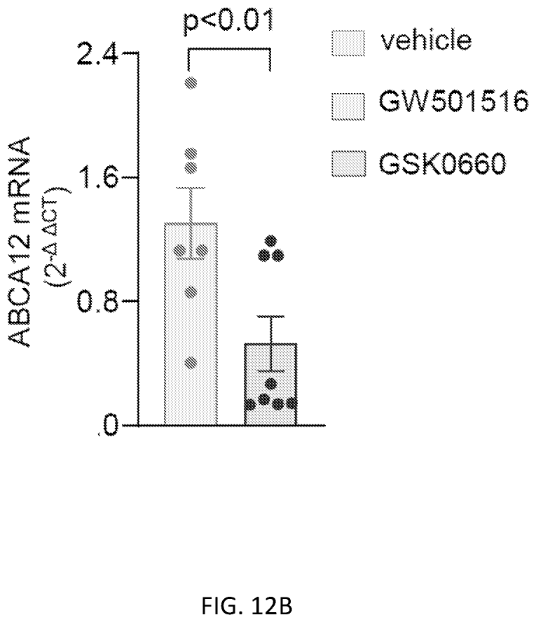

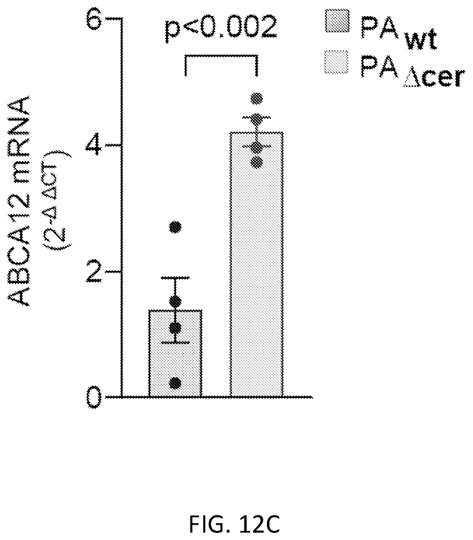

Subjects participating in the study were chronic wound patients undergoing negative pressure wound therapy (NPWT) as part of standard clinical care. Demographic characteristics of patients and wound-related information are presented in FIG. 12I. The NPWT dressing (sponges) were collected from each patient for cell isolation and wound fluid collection. Wound fluids were derived from NPWT dressing by lavaging the wound dressing with saline solution. Declaration of Helsinki protocols were followed, and patients gave their written informed consent. Levels of cyclic diguanylate (c-di-GMP) in the human wound fluid was measure using c-di-GMP ELISA kit (MyBioSource, Inc., San Diego, CA) as per manufacturer's instructions and normalized to albumin levels measured using human albumin ELISA Kit (Abcam). C-di-GMP is a marker of PA biofilm infection, therefore, wound fluids rich in c-di-GMP were used for lipidomic analysis.

Lipidomic Analysis of Human would Fluid Samples.

Wound fluid samples were centrifuged to remove any debris and cells, then processed as described for plasma. Lipids were extracted from samples in methanol:dichloromethane in the presence of internal standards. The extracts were concentrated under nitrogen and reconstituted in 0.25 mL of 10 mM ammonium acetate dichloromethane:methanol (50:50). The extracts were transferred to inserts and placed in vials for infusion-MS analysis, performed on a Shimazdu LC with nano PEEK tubing and the Sciex SelexIon-5500 QTRAP. The samples were analyzed via both positive and negative mode electrospray (analyses were done by Metabolon®). The 5500 QTRAP scan was performed in MRM mode with the total of more than 1,100 MRMs. Individual lipid species were quantified by taking the peak area ratios of target compounds and their assigned internal standards, then multiplying by the concentration of internal standard added to the sample. Lipid class concentrations were calculated from the sum of all molecular species within a class, and fatty acid compositions were determined by calculating the proportion of each class comprised by individual fatty acids.

### Lipidomic Analysis of Skin Ceramides Using LC ESI-MS/MS.

Targeted analysis of the sphingolipidome was undertaken. Briefly, 500 pmol of each sphingolipid internal standard mixture (Avanti) were spiked into the porcine wound edge tissue and lipids were via a modified Bligh-Dyer protocol. The resultant mixture was sonicated to disperse aggregates, centrifuged to remove particulate and the cleared supernatants were transferred to a new glass tube, dried down, and reconstituted in methanol (600 μl). Extracted lipids were separated using a Kinetix C18 column (50×2.1 mm, 2.6p) (Phenomenex) on a Shimadzu Nexera, ultra-performance liquid chromatography system and eluted using a linear gradient (solvent A, 58:41:1 CH3OH/water/HCOOH 5 mm ammonium formate; solvent B, 99:1 CH3OH/HCOOH 5 mm ammonium formate, 20-100% B in 3.5 min and at 100% B for 4.5 min at a flow rate of 0.4 ml/min at 60° C.). Electrospray ionization with tandem mass spectroscopy using a QTRAP 6500 instrument (SCIEX) was used to detect and quantify sphingolipids. Individual sphingolipids were monitored using precursor→product MRM pairs. The parameters for detection of sphingolipids via QTRAP 6500 mass spectrometry system are as follows: Curtain Gas: 30; CAD: High; Ion Spray Voltage: 5500V; Source Temperature: 500° C.; Gas 1: 40; Gas 2: 60; Declustering Potential: 80V. Collision Energies varied per transition. MRM transitions and collision energies varied depending on the analyte investigated.

Untargeted lipidomics was undertaken as previously published. Briefly, the different bacteria were cultured for 6 hours in 3 ml Tryptic Soy Broth (TSB) in snap cap tubes. All bacterial spent media samples were diluted 1:1 with sterile phosphate buffered saline (PBS) prior to performing lipase assays. Heat inactivated controls of the spent media samples were prepared by heating the supernatant to 80° C. in a heat block for 20 minutes prior to performing 1:1 dilution used in the assays. Lipids were prepared by, taking 0.6 mg (500 μl of 1.25 mg/ml stock) of total heart extract dried under vacuum in a speedvac for 30 minutes. Lipids were extracted from this mixture via a modified Bligh and Dyer method. Lipids from the 250 μl reaction volume were extracted using 1 ml methanol and 0.5 ml of chloroform followed by addition of 10 μl of SPLASH lipidomics internal standard mix (Avanti Polar Lipids) and incubated at room temperature for 10 minutes. From the phase-separated mixture, the bottom hydrophobic organic phase (bottom phase) was transferred into a fresh glass tube. The organic phase containing the lipids was dried under a stream of nitrogen and lipids were resolubilized in 250 μl of the infusion solvent (MeOH/CH2Cl2, 50:50 containing 5 mM ammonium acetate). Finally, lipids were analyzed via the use of MS/MSALL approach as previously disclosed. Briefly, MS/MSALL was performed using a Sciex Triple-TOF 5600+ via direct infusion using a Shimadzu SIL-20ACXR at a flow rate of 9 ul/min over 25 minutes.

The samples were infused using a flow gradient. The experimental parameters used to analyze lipids in the mass range (m/z 100-1200) were positive ionization mode: CUR 30, GS1 16, GS2 40, TEM 150, accumulation time 5000 ms, CAD 4, CE 38 CES 5, DP 80 and negative ionization mode: CUR 30, GS1 16, GS2 40, TEM 100, accumulation time 3000 ms, CAD 4, CE-45, CES 5, DP-80. Data analysis was performed as previously described and the ceramide content was reported and mol % values.

### Lipidomic Analysis of Sphingosine-1-Phosphate (S-1-P). Sample Preparation:

Samples were weighted in Precellys tubes and spiked with 20 μl of IS ceramide/sphingolipid mixture I (LM-6002 Advanti Polar Lipids, USA) with 0.5 nmol of d17:1-P. 200 μl of water were added for homogenization. The total volume was collected and transferred to a glass vial and 750 μL of methanol/chloroform in a 2:1 (v/v) ratio was added. The mixture was sonicated and then incubated overnight at 48° C. in a heating block. After cooling down at room temperature, 75 μL of 1M KOH in methanol was added to the samples, then sonicated and incubated for two hours at 37° C. in a heating block. After cool down, samples were centrifuged at 13,000 g for 8 minutes and the supernatant was transferred to a fresh microcentrifuge tube and vacuum concentrated to dryness. The extract was reconstituted in 200 μL of 80:20 mobile phases A/B. Then sonicated for 5 minutes, centrifuged at 13,000 rpm for 8 minutes, and the supernatant was transferred to HPLC vials. HPLC/MS-MS Analysis: S-1-P levels were quantitated by HPLC/MS-MS. Separation was performed on an Agilent Rapid Res 1200 HPLC system using an Xterra C18 (2.1×150 mm, μm) column. Mobile phase A is 10 nM of ammonium formate and 0.1% formic acid and mobile phase B is 10 nM of ammonium formate in 1:1 (v/v) of Isopropanol:acetonitrile plus 0.1% formic acid. Initial conditions were 85:15 A:B, followed by a linear gradient to 0:100 at 8 min, and held until 20 min. Column re-equilibration was performed by returning to 85:15 A:B at 21 minutes and held until 30 minutes. Column flow rate was 0.3 mL/min. Retention time for S-1-P was 11.3 minutes.

### Analysis:

Analytes were quantified by MS/MS utilizing an Agilent 194 6460 triple quadrupole mass spectrometer with electrospray ionization (ESI). Quantitation was based on Multiple Reaction Monitoring (MRM). ESI positive mode was used with a transition of 380.4 to 264.4 with a collision energy (CE) of 30 V for d18:1-P base and a transition of 366.4 to 250.4 with CE of 30 V for the internal standard d17:1-P. A fragmentor energy of 135 V and a dwell time of 80 ms was used. Source parameters were as follows: nitrogen gas temperature=325° C. and flow rate=10 L/min, nebulizer pressure=50 psi, sheath gas temperature=250° C., sheath gas flow rate=7 L/min, and capillary potential=3.5 kV. All data were collected and analyzed with Agilent MassHunter B.03 software. Quantitation was based on a 5 point standard curve, with concentration range from 0.25 to 2500 ng/mL, by spiking d18:1-P and d17:1-P base into PBS due to the presence of endogenous molecule. Standard curves were fit to a linear function and the correlation coefficients>0.99 were obtained. The S-1-P assay method was adapted from Merrill.

Next Generation Sequencing for Bacterial 16S rRNA.

Porcine wound edge tissue was pulverized using a tissue pulverizer (6770 Freezer/Mill). Microbial DNA in each sample were sequenced by MicrogenDx Inc using the Illumina MiSeq sequencer. Forward and reverse primers were used to detect and amplify the target sequence, for 16S gene in bacteria. The samples were differentiated from each other when run on the MiSeq sequencer by a “tag,” a unique identifying sequence attached to the forward and reverse primers implemented when the targeted sequence is amplified using PCR. Following PCR, purification of the pooled DNA was done by removing small fragments using both Agencourt Ampure beads and Qiagen Minelute kit. The DNA was quantified and prepared for sequencing. Finally, the DNA library is run on the MiSeq sequencer. The sequencing reads were analyzed for quality and length during the data analysis. The data analysis pipeline consisted of two major stages, the denoising and chimera detection stage and the microbial diversity analysis stage. During the denoising and chimera detection stage, denoising was performed using various techniques to remove short sequences, singleton sequences, and noisy reads. With the low-219 quality reads removed, chimera detection was performed to aid in the removal of chimeric sequences. Any read that fell below the quality score or quality metric or appropriate length were discarded. The high-quality sequencing reads of the variable region of 16S rRNA were compared to curated database of MicrogenDx. The database is comprised of 18500 unique bacteria.

### Cell Culture.

Human keratinocytes were grown under standard culture conditions (at 37° C. in a humidified atmosphere consisting of 95% air and 5% CO2) in DMEM growth medium supplemented with 10% FBS, 100 IU/ml penicillin, 0.1 mg/ml streptomycin, 10 mmol/l L-glutamine.

### In Vitro Human Keratinocytes-Pseudomonas Biofilm Co-Culture.

Bacterial biofilms were co-cultured with human keratinocytes cells following the method adapted from Anderson. The confluent cultures of human keratinocytes cells were inoculated with bacterial cultures of PAwt or PAΔcer (105 CFU/ml) in antibiotic free culture media. The plates were incubated for 1 h at 37° C. and 5% CO2. Post 1 h, fresh DMEM (without antibiotics) supplemented with 0.4% arginine was added to the plate. Post 7-8 hours, cells were harvested. The co-culture media was filtered using 0.2 μm filters and used for ceramidastin experiment. Human keratinocytes were treated with conditioned media (25% v/v) of cells co-cultured with PAwt biofilm containing ceramidastin (10 μg/ml) for 24 h.

### In Vitro Pseudomonas Biofilm Model.

In vitro biofilm culture on polycarbonate membrane was adapted from Zhao (Wound Repair Regen 2010; 18(5):467-77). In vitro biofilm was grown on a polycarbonate membrane (PCM) with or without porcine skin lipids for 24 h. Post 24 h of inoculation the biofilm was processed for SEM, mRNA using real-time qPCR or for wheat germ agglutinin (WGA staining).

Isolation of lipids from porcine skin. 4 cm×4 cm×0.25 cm (height/width and depth) section of porcine skin obtained from back of the pig was cut into chunks no larger than 3 mm³ using a pair of surgical scissors and a scalpel. The tissue thus obtained was sonicated using a probe sonicator (QSonica Q 500). Care was taken to maintain the tissue and the sonicated homogenate at a temperature at or below 40° C. during the process. Following this process of homogenization lipids were extracted from a 400 μL of the homogenate using a modified Bligh and Dyer method. Briefly 1 mL of methanol was added to the homogenate followed by batch sonication. Thereafter 0.5 ml of Chloroform was added to the mixture followed again by sonication. The mixture thus obtained was incubated at 480° C. for 4 hours to enable the extraction of the highly hydrophobic lipid species of the stratum corneum. The mixture was centrifuged at 6000×g for 15 minutes to pellet out the debris and the extract thus clarified was transferred to a new glass vial. Thereafter an additional 1 mL of chloroform was added to the mixture followed by vortexing for 10 seconds. 2 mL of water was then added to this mixture to separate the hydrophobic and hydrophilic phases. The mixture was vortexed again and centrifuged at 6000×g for 15 minutes to better enable the separation of phases. Using a glass Pasteur pipet, the bottom organic layer was carefully removed and transferred to a new glass via. The remaining extract was re-extracted with 1 mL of chloroform, centrifuged and the new bottom organic phase was combined with the organic phase of the first extract. The hydrophobic lipid extracts thus obtained were dried via a vacuum concentrator without the use of heat. The dried lipid extract was then reconstituted in 500 μL of 1:1 methanol:dichloromethane and was used in the subsequent studies.

Depletion of Lipids from Porcine Skin.

Porcine skin tissue was pulverized using cryo-pulverizer. 1 g of porcine tissue slowly added to PBS (10% w/v ratio). The mixture was homogenized using a probe sonicator at 4° C. 400 μL of the homogenate was taken and 1 ml of methanol was added followed by bath sonication. 0.5 ml chloroform was added to the mixture followed again by sonication. Incubation of mixture was done overnight at 48° C. Next day the mixture was centrifuged at 4300 rpm for 20 mins. The extract thus clarified transferred to a new glass vial. Additional 1 mL of chloroform was added to the mixture followed by vortexing for 10 s. To separate the hydrophobic and hydrophilic phases, 2 ml of water was added to this mixture. The mixture was vortexed again and centrifuged at 4300 rpm for 20 mins to better enable the separation of phases. Using a glass Pasteur pipet, the top layer was carefully removed and transferred to a new glass vial. The remaining extract was re-extracted with 1 mL of chloroform, centrifuged and the new top phase was removed and combined with the first extract.

### Lipid Quantification Assay.

The depletion of lipids was quantified using Lipid Assay Kit (neutral lipids) (abcam), Lipid Assay Kit (unsaturated fatty acids) (abcam) as per manufacturer's manual.

### Laser Capture Microdissection (LCM).

Frozen tissue blocks were cut into 8-μm sections. The sections (2-3) were mounted on each RNAZap-treated thermoplastic (polyethylene napthalate)-covered glass slide (PALM Technologies, Bernreid, Germany) and kept at −80° C. until use. When needed, sections were thawed and fixed for 1 min in one of the following fixatives: 95% ethanol, 10% neutral-buffered formalin, or acetone and catapult. Epidermal layers of biofilm infected and control wound tissues were captured in lysis buffer provided with Cells-Direct RNA kit (Invitrogen). This was followed by RNA extraction and reverse transcription and mRNA quantification using real-time PCR.

### Immunohistochemistry and Immunocytochemistry.

OCT embedded wound tissue blocks were sectioned using a cryostat. Immunohistochemical staining of the frozen sections were performed using the following primary antibodies: anticeramide (Enzo Life Science, MID 15B4; dilution 1:100), α-PPARS (Abcam, dilution: 1:200), α-ABCA12 (Abcam, dilution: 1:200). To enable fluorescence detection, sections were incubated with appropriate Alexa Fluor® 488 (green, Molecular probes), Alexa Fluor® 564 (red, Molecular probes) conjugated secondary antibodies. The sections were counterstained with DAPI (Sigma). For immunocytochemistry, cells were fixed with IC fixation buffer (eBioscience), blocked with 10 percent normal goat serum (Vector Laboratories), incubated with primary and secondary antibodies and counterstained with DAPI. Mosaic images were collected using collected using a Zeiss Axiovert 200 M, inverted fluorescence microscopy or confocal microscopy (LSM880). Image analysis was performed using Zen (Zeiss) software to quantitate fluorescence intensity (fluorescent pixels).

### Hematoxylin & Eosin (H&E) Staining.

The OCT sections were fixed in chilled acetone for 3 mins. The sections (10 μm thick) were stained with hematoxylin and eosin (H&E). The OCT sections were fixed with acetone then washed with water. The sections were stained with hematoxylin for 5 m, washed in water and counterstained with eosin for 2 m. The sections were dehydrated with graded alcohol and washed with xylene followed by mounting.

### Nile Red Staining.

Nile red staining method was adapted from Greenspan. Stock solution (1 mg/ml) of Nile red (Thermo Scientific) in acetone was prepared. The OCT wound tissue sections were washed by PBS and stained with Nile red solution (1:100 dilution) for 10 mins. The sections were washed in PBS, stained with DAPI and mounted.

### Wheat-Germ Agglutinin (WGA) Staining.

The PCM disc with bacteria biofilm were transferred from the agar plates to glass slides. Wheat Germ Agglutinin, Alexa Fluor™ 488 Conjugate (Invitrogen) 1 mg/ml stock solution was diluted in PBS. The PCM discs were stained with Wheat Germ Agglutinin (dilution 1:200) for 10 mins. The PCM discs were then mounted and imaged Zeiss LSM 880 microscope equipped with the AIRYscan detector.

### Quantitative RT PCR.

Porcine wound tissue was pulverized using tissue pulverizer (6770 Freezer/Mill) and total RNA was extracted using miRVana (Thermo Fisher Scientific). cDNA was made using SuperScript™ III First-Strand Synthesis System (Invitrogen) or SuperScript™ VILO™ cDNA Synthesis Kit (Invitrogen). Quantitative or real-time PCR (Taqman or Sybr Green) approach was used for mRNA quantification.

### Western Blot Analyses.

Western blot was performed using antibodies against anti-PPARδ (Thermo Scientific, 1:500), anti-CerS3 (Origene, 1:500). β-actin (Sigma Aldrich, 1:5000) was used as housekeeping.

### PPARδ Trans-Activity Assay.

Human keratinocytes cells were treated with 5 μmol of long chain ceramides, C18:0 and C24:0 (Avanti Polar Lipids Inc., Alabama) using Lipofectamine LTX (Thermo Fisher Scientific). Cells were collected 48 h post transfection and nuclear protein was extracted using Nuclear Extraction Kit (RayBiotech) as per manufacturer's protocol. PPARδ trans-activity was measured using PPAR delta Transcription Factor Assay Kit (abcam) as per manufacturer's instructions.

### DNMT3B Activity Assay.

Human keratinocytes cells were treated with 5 μmol of Sphingosine-1-phosphate (Avanti) for 48 h. Cells were collected and DNMT3B activity was measured using EpiQuik DNMT3B Activity/Inhibitor Screening Assay Core Kit (EPIGENTEK) according to manufacturer's instructions and using recombinant DNMT3B protein, (ACTIVE MOTIF) as a positive control for the assay.

### PPARδ Promoter Assay.

PPARδ promoter assay was done as described previously. mNP-Luc (PPARdelta promoter) was a gift (Addgene plasmid #16532; http://n2t.net/addgene:16532; RRID:Addgene_16532. Human keratinocytes cells were co-transfected with the PPARδ promoter reporter construct and treated with 5 μmol of long chain ceramides, C:18 and C:24 (Avanti Polar Lipids Inc., Alabama) using Lipofectamine™ LTX Reagent with PLUS™ Reagent (Thermo Fisher Scientific). Media were collected 48 h post transfection and the secreted GLuc and SEAP activities were measured with the Secrete-Pair Dual Luminescence Assay Kit (GeneCopoeia) according to the manufacturer's protocol using the Berthold Luminometer (Berthold Technologies). Normalized promoter activity has been presented as the ratio of GLuc to SEAP activities.

miR-Target 3′-UTR Luciferase Reporter Assay.

HaCaT keratinocytes were transfected with miRIDIAN mimic-miR-106b followed by transfection with miR target PPAR6-3′-UTR plasmid (NM_006238, Genecopoeia HmiT105061-MT06) or CerS3-3′-UTR plasmid (NM_178842, Genecopoeia HmiT095486-MT06). Luciferase assay and normalization was performed using the dual-luciferase reporter assay system (Promega). Normalization was achieved by co-transfection with Renilla plasmid. Data are presented as the ratio of firefly:renilla luciferases.

miRNA Delivery to Human Keratinocytes.

Transfection of human keratinocytes cells was performed as described.

Briefly, keratinocytes were seeded in antibiotic-free DMEM medium 24 h before transfection. DharmaFECT 1 transfection reagent (Dharmacon; Lafayette, CO) was used to transfect cells with 100 nM miR-106b, (Dharmacon; Lafayette, CO). Transfection of non-targeting miRNA negative controls was performed for the control groups. Cells were harvested/reseeded after 72 h.

### Bisulfite Sequencing for PPARδ Promoter.

The PCR products were gel extracted (GenElute Gel Extraction Kit, Sigma, cat #NA1111-1KT) and Sanger sequencing was done using ABI 3730 Genetic Analyzer. Analysis was done using Sequencher 5.4.5 (Gene Codes Corporation, MI). Results were confirmed by cloning the purified DNA into pGEM-T Easy Vector System II (Promega, cat #A1380), followed by sequencing.

### Bisulfite Conversion of DNA.

Bisulfite conversion of DNA from HaCaT keratinocytes were transfected with Sphingosine-1-phosphate (5 μM, 48 hours) was performed using the Cells-to-CpG Bisulfite Conversion Kit (Thermo Fisher Scientific, part number: 4445555), as per manufacturer's protocol. The cells were lysed, and DNA was denatured using the denaturation reagent in a PCR tube at 50° C. for 10 min. Unmethylated cytosines were converted to uracil in the denatured DNA samples via treatment with conversion buffer containing bisulfite in a PCR reaction under following conditions: (1) 65° C. for 30 min, (2) 95° C. for 1.5 min, (3) 65° C. for 30 min, (4) 95° C. for 1.5 min, (5) 65° C. for 30 min, and (6) 4° C. up to 4 hr. This was followed by the removal of salts and desulfonation of the converted DNA in a binding column, a series of washing steps, and then elution of DNA. The converted DNA was stored at 20° C. until further used.

### Quantification and Statistical Analysis.

The data analysis was performed using student's t-test (two-tailed) presented as mean±SEM. Mean, SEM and student paired t-test analysis was done using in-built function in Microsoft Excel 2010. Comparisons among multiple groups were tested using ANOVA in-built function in GraphPad Prism 8.4.2. p<0.05 was considered statistically significant.

### Results

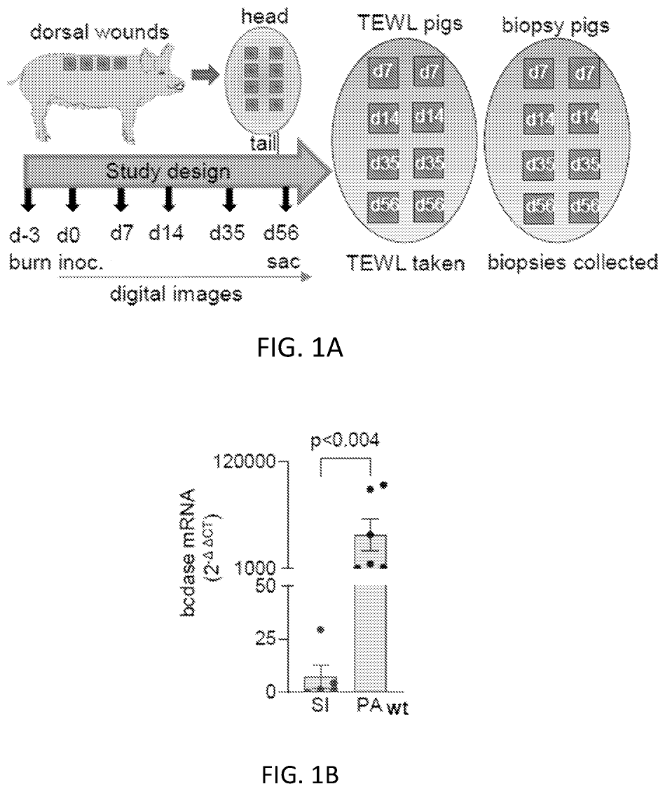

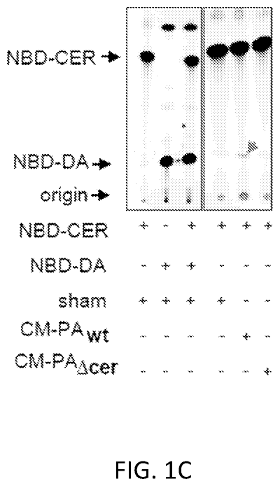

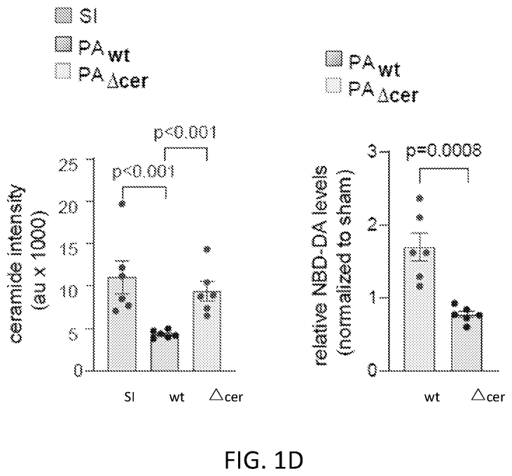

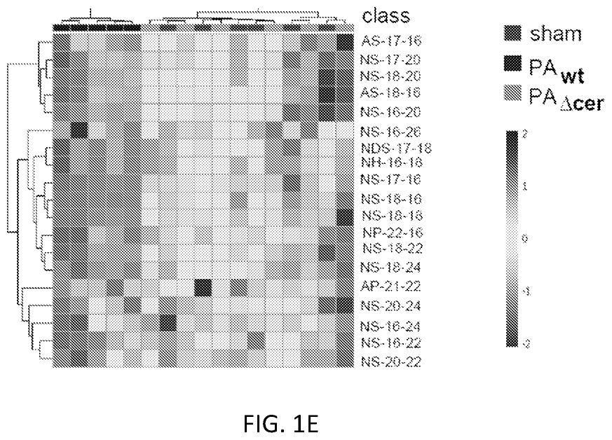

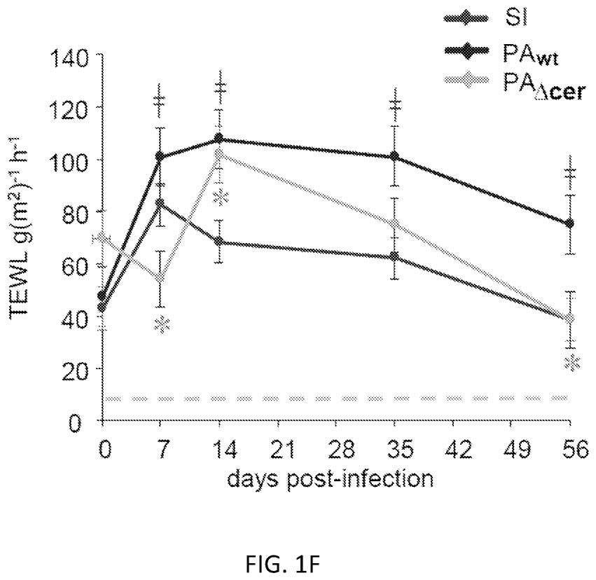

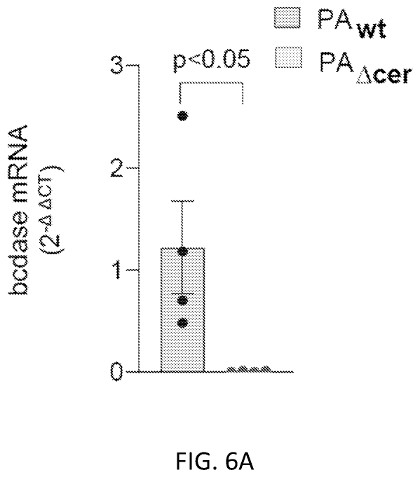

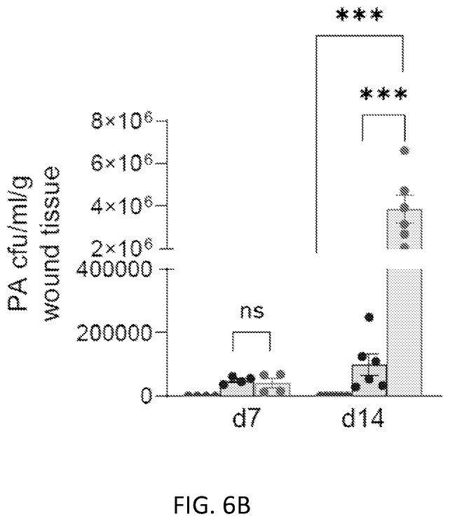

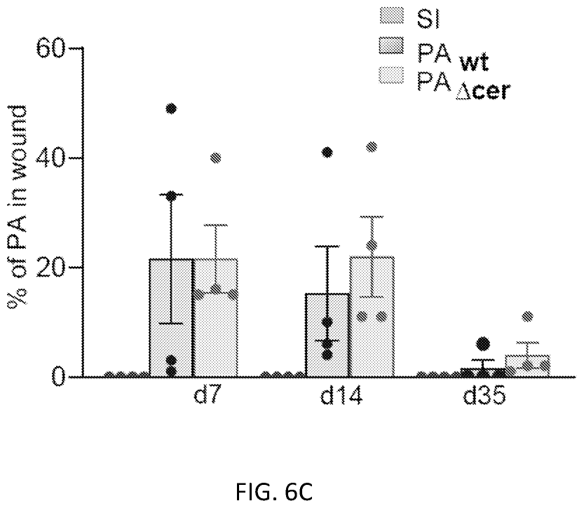

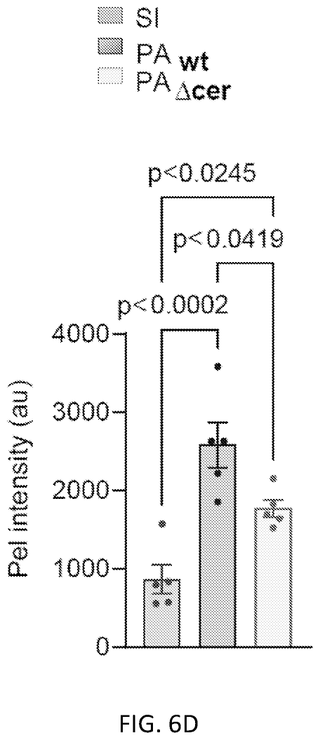

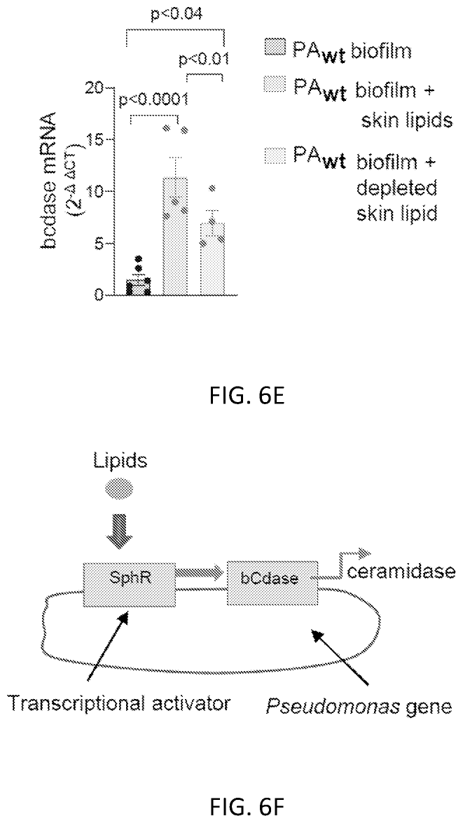

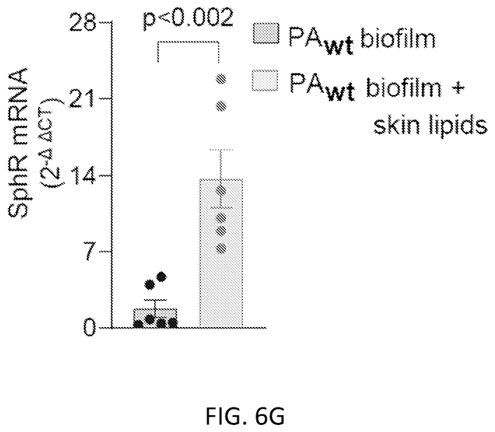

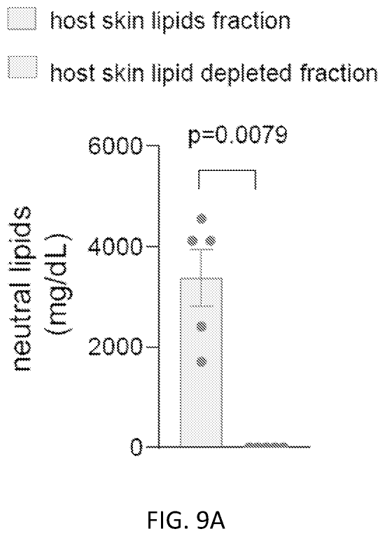

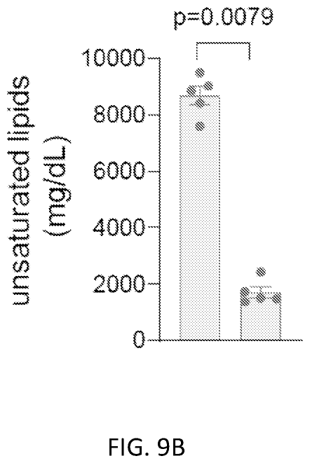

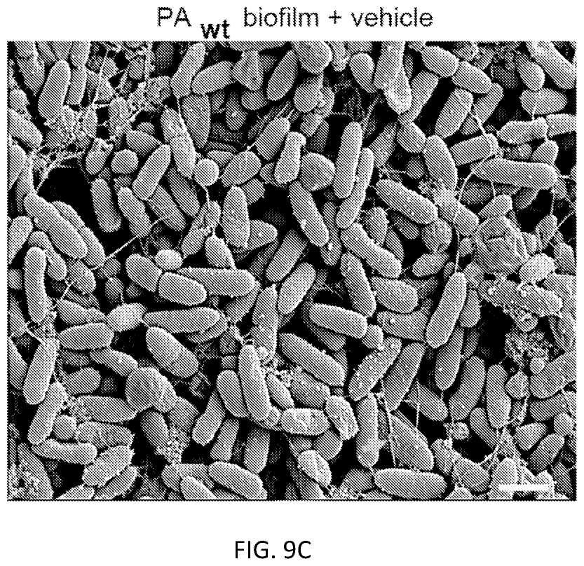

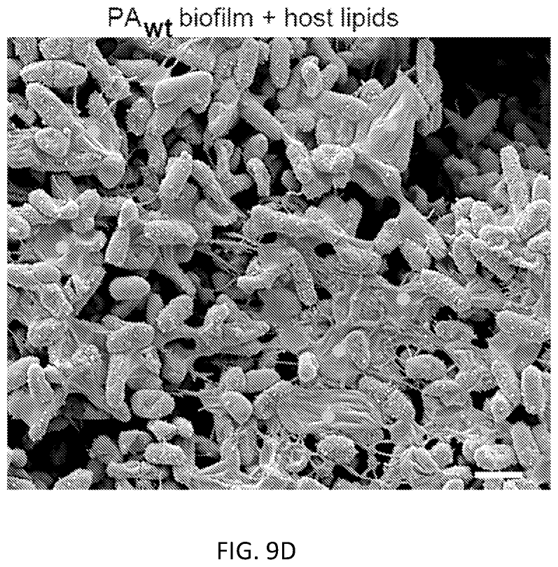

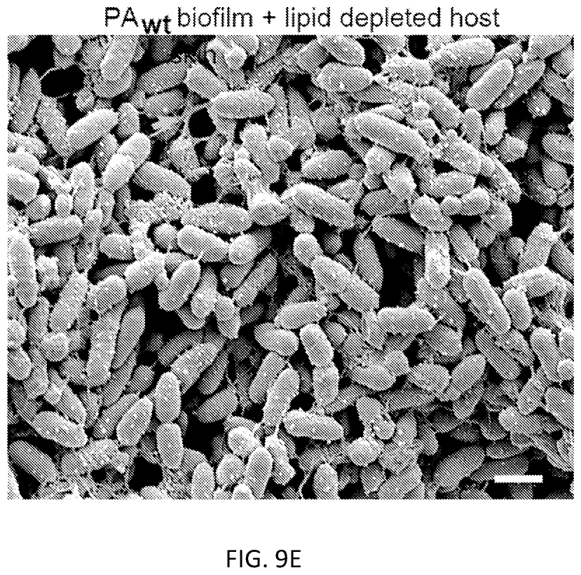

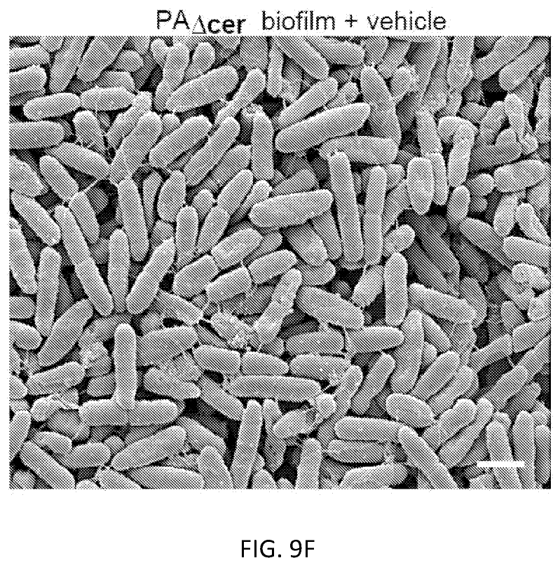

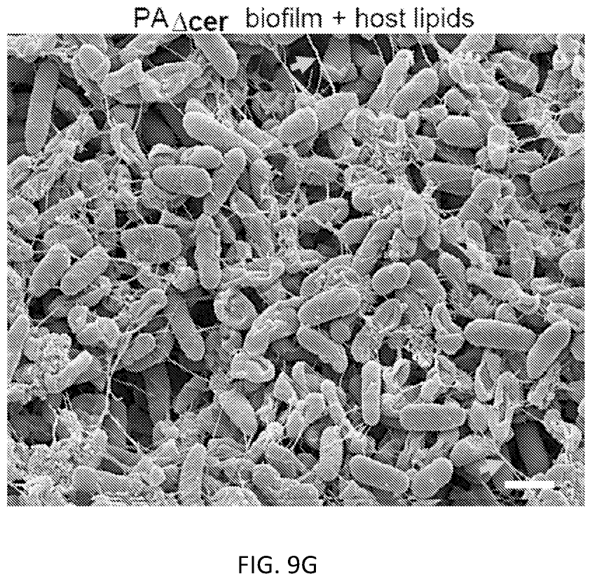

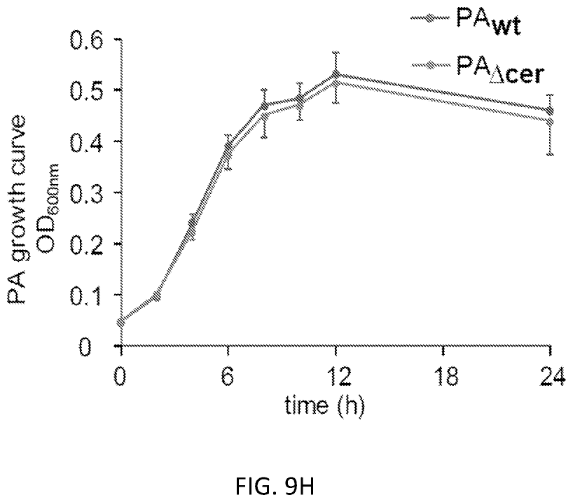

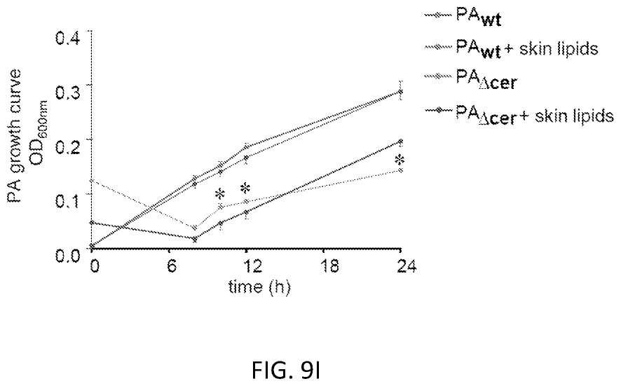

Wound infection and biofilm formation depletes host skin ceramides. In an established pre-clinical porcine chronic wound biofilm model (FIG. 1A), the expression of PA ceramidase was induced by three orders of magnitude on day 7 following infection with PAwt (FIG. 1B). Control wounds were not subjected to induced infection and were allowed to be colonized by natural skin microflora. These wounds are referred to as spontaneously infected (SI). Bacterial ceramidase is known to cause breakdown of host ceramides. To determine the significance of PA ceramidase a ceramidase deficient PA strain (PAΔCer) was studied. The loss of ceramidase (bcdase) gene in the mutant bacterial strain was validated using qRT PCR (FIG. 6A). Loss of ceramidase activity was evident in PAΔCer as measured by thin layer chromatography (TLC) using a fluorescent ceramide analog (FIG. 1C). PA infection was confirmed by CFU assay (FIG. 6B). To identify bacterial species and their abundance, a 16S rRNA (variable region) next generation sequencing (NGS) was performed. PA infection was further confirmed on the basis of NGS sequencing (FIG. 6C). Though the initial infection was polymicrobial (PA+AB), overtime PA became the dominant species in biofilm (FIG. 6C). The formation of bacterial biofilm aggregates was validated using scanning electron microscopy (SEM) and staining with PA biofilm matrix component specific antibody pel (FIG. 9C-9G).

TABLE 2

| | | catalogue | dilution |
|---|---|---|---|
| | company | number | used |
| antibody | | | |
| anti- ceramide | Enzo | MID 15B4 | 1/100 |
| α-PPARδ | Abcam | ab23673 | 1/200 |
| α-ABCA12 | Abcam | ab154037 | 1/200 |
| Alexa Fluor ® 488 | Molecular probes | A32731 | 1/200 |
| Alexa Fluor ® 568 | Molecular probes | A-11031 | 1/200 |
| anti-PPARδ | Thermo Scientific | PA1-823A | 1/500 |
| anti-CerS3 | Acris | AP16822PU-N | 1/500 |
| anti-β-actin | Sigma Aldrich | A-5441 | 1/5,000 |
| stain | | | |
| DAPI | Sigma | D9542 | 1/10,000 |
| Nile red | Thermo Scientific | N1142 | 10 μg/ml |
| Wheat-germ agglutinin | Invitrogen | | 5 μg/ml |

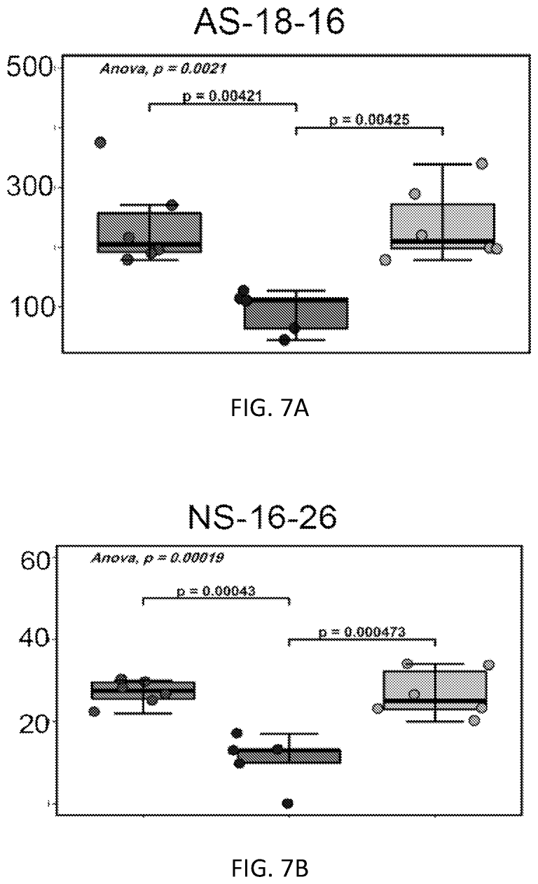

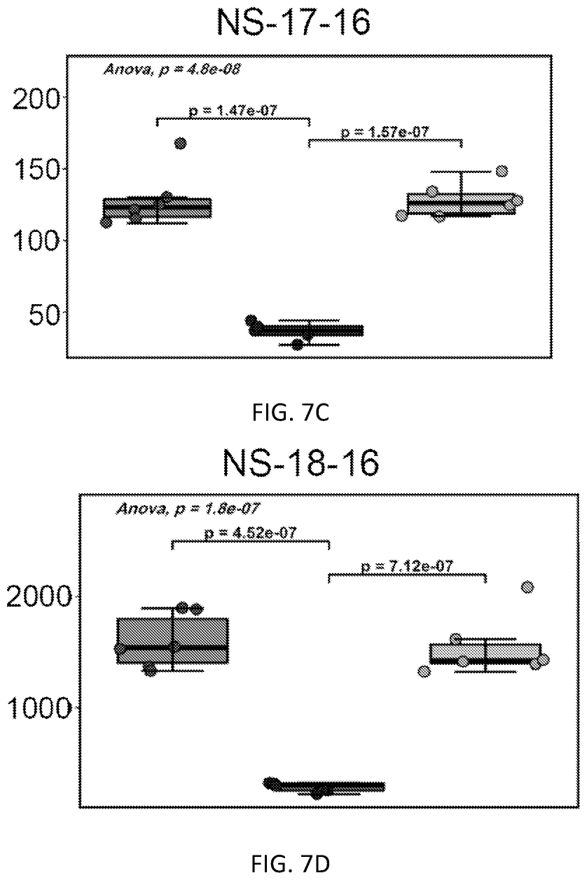

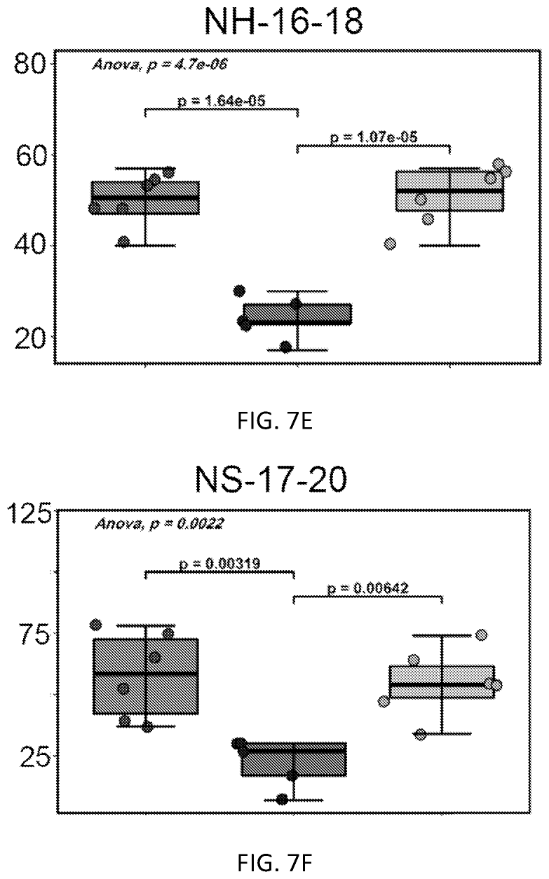

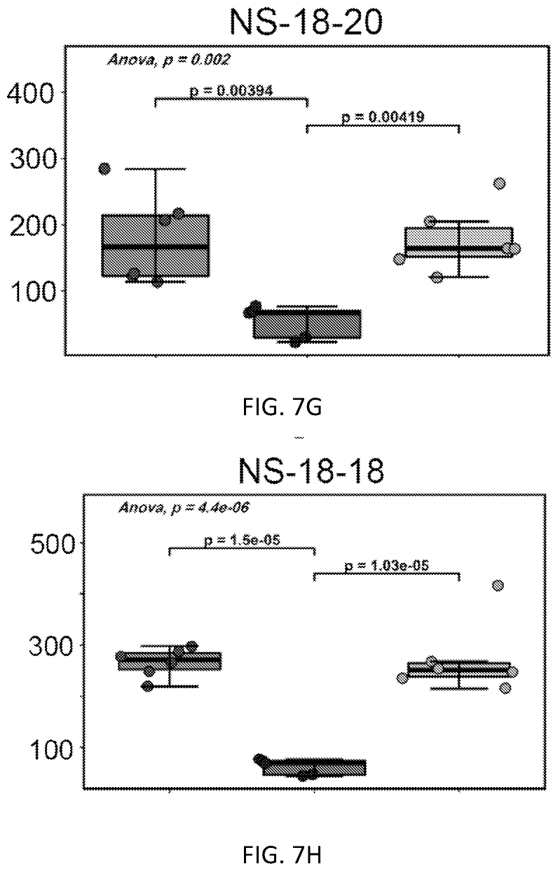

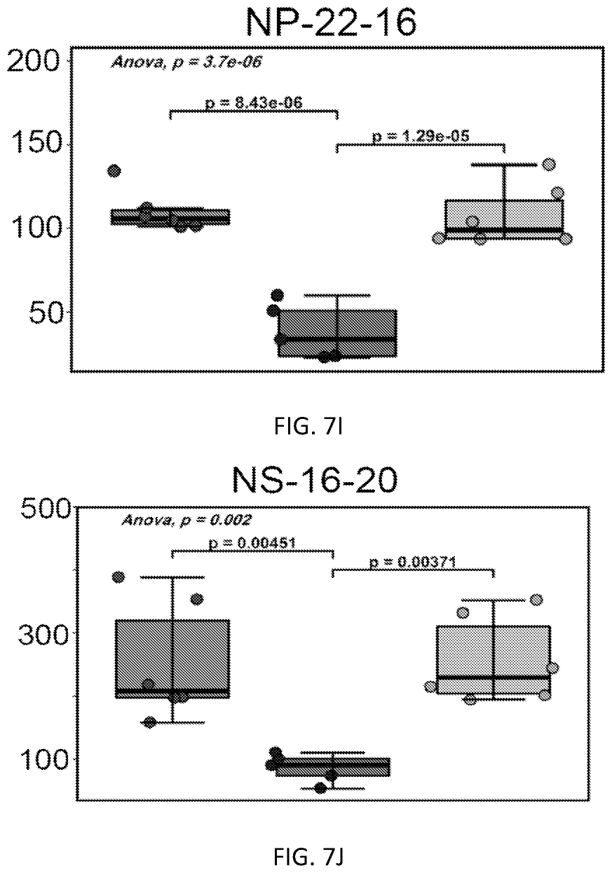

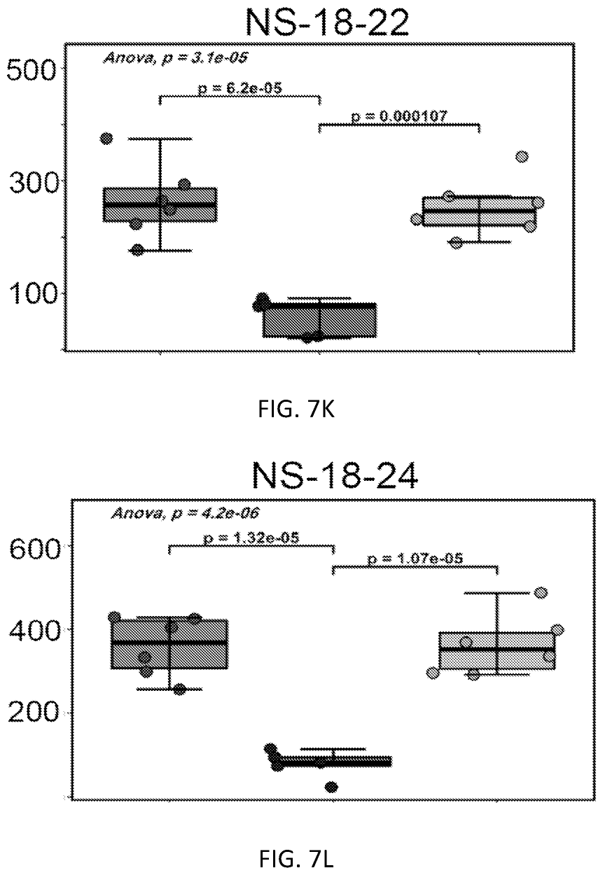

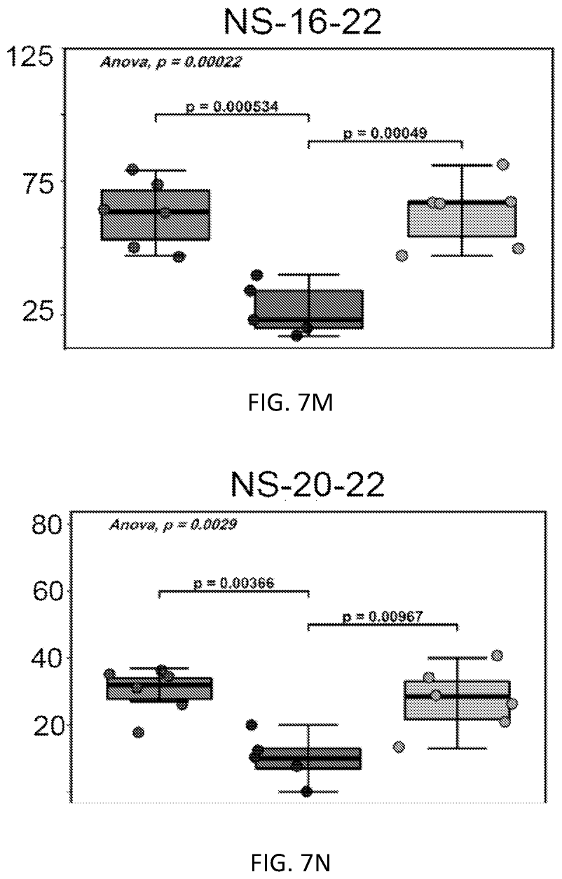

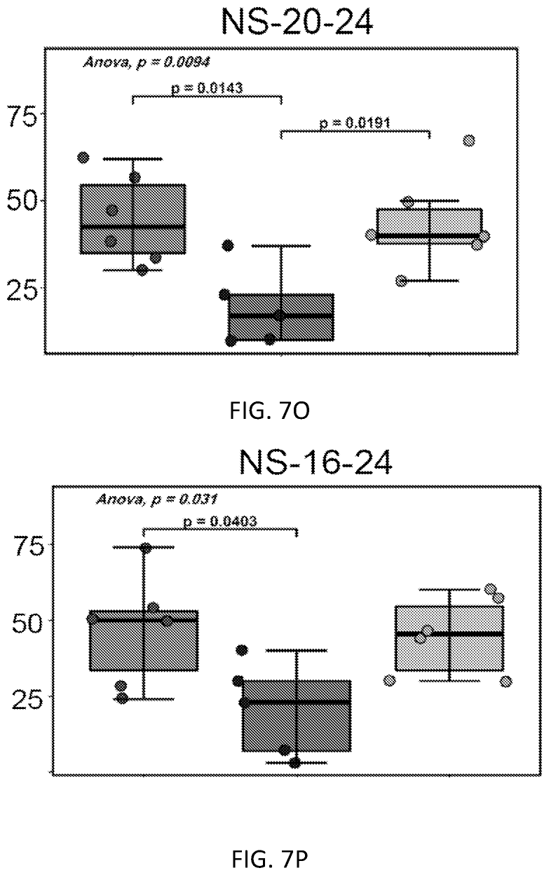

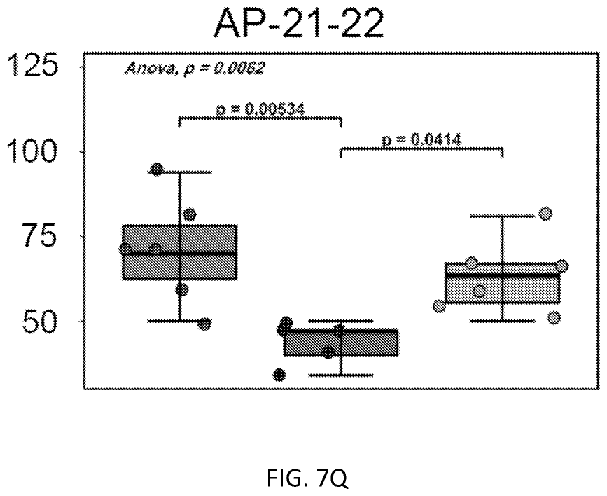

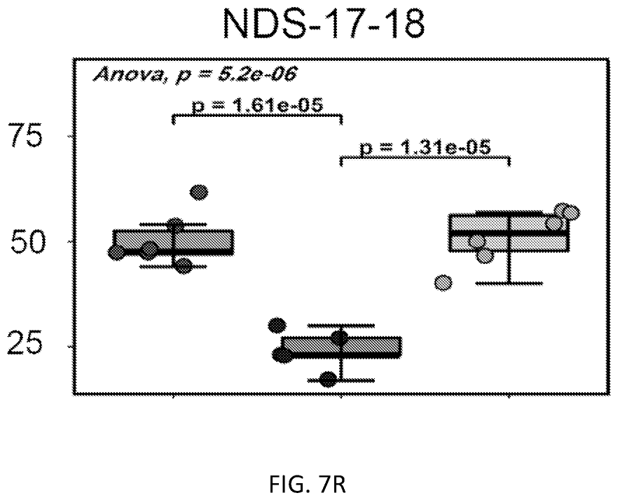

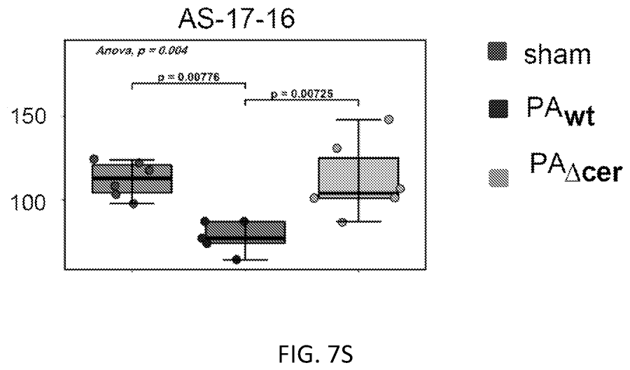

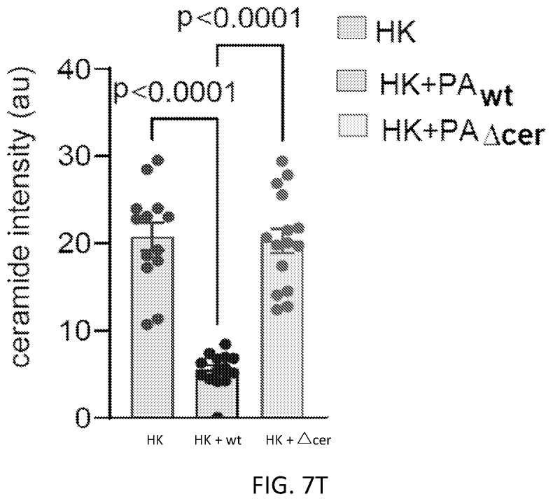

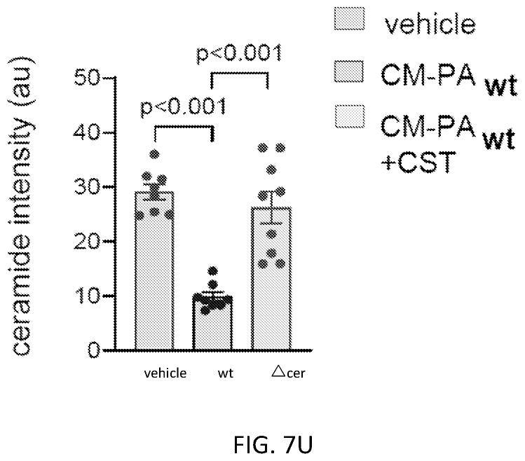

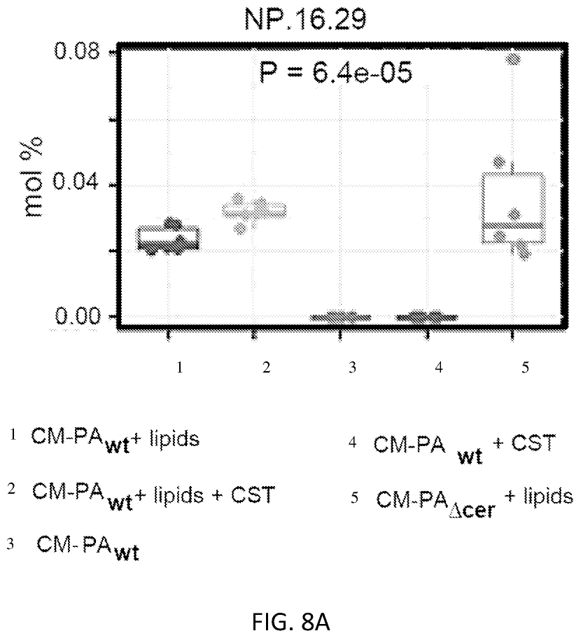

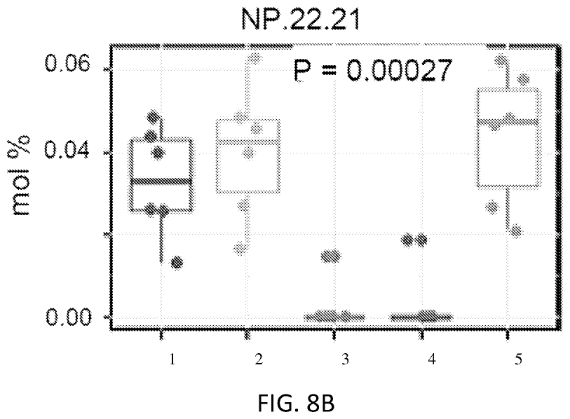

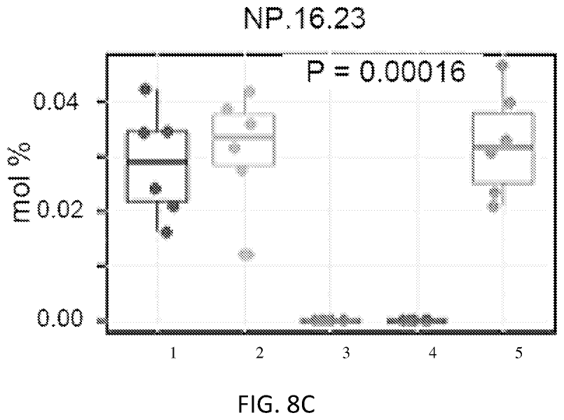

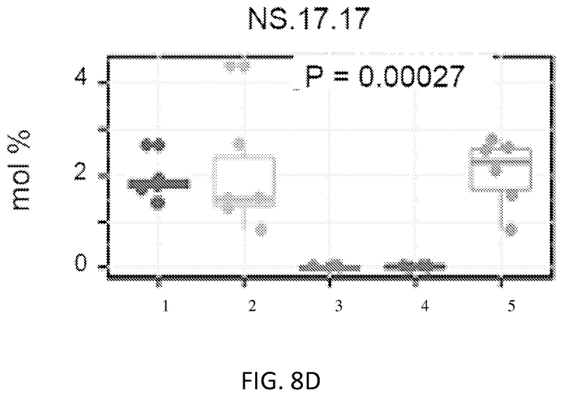

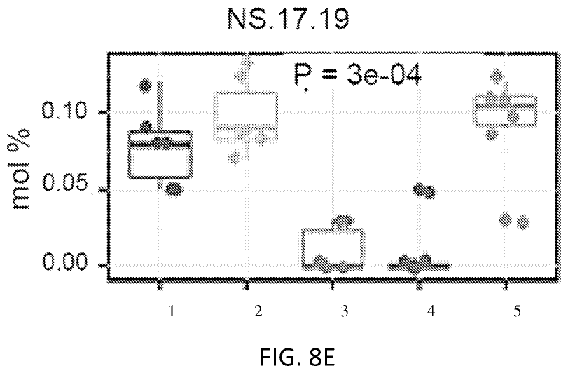

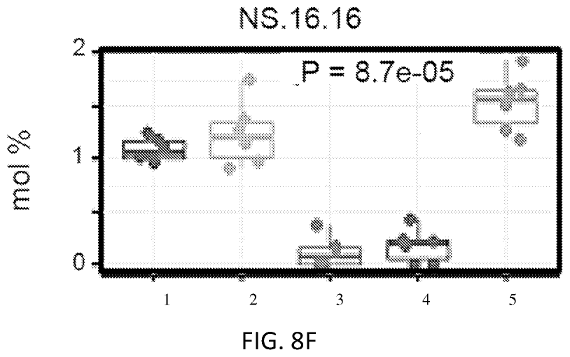

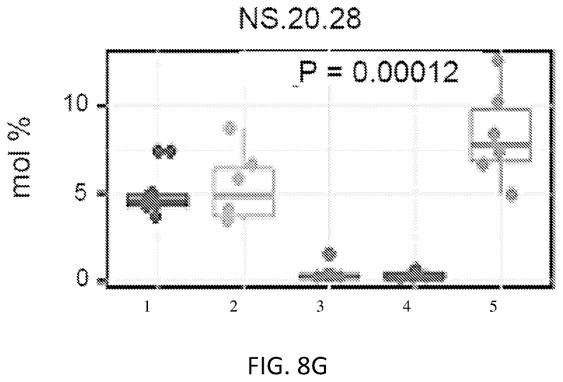

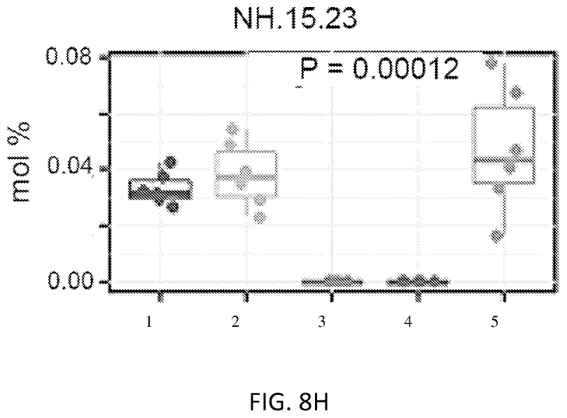

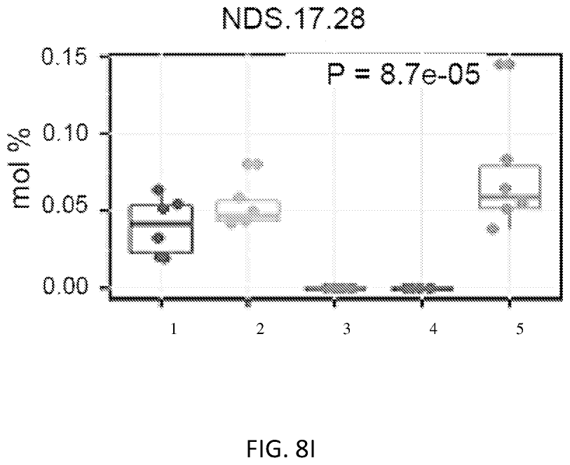

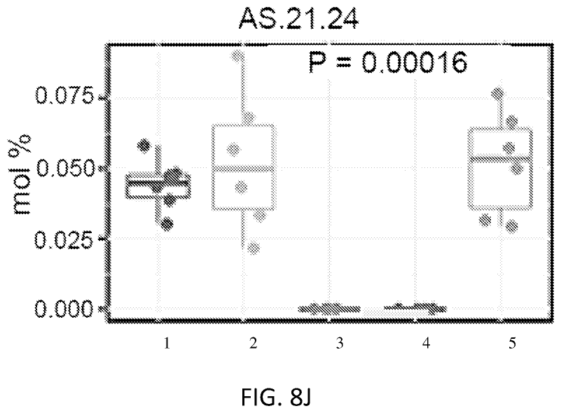

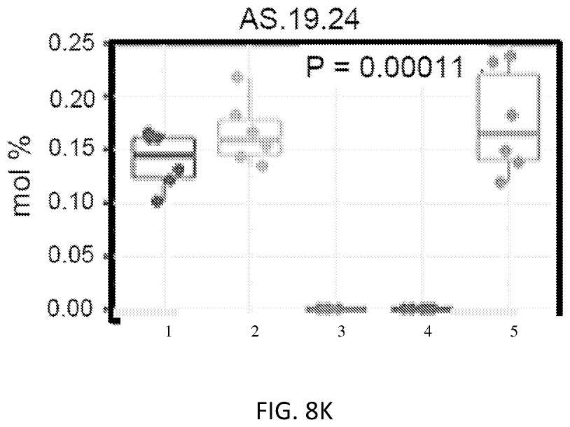

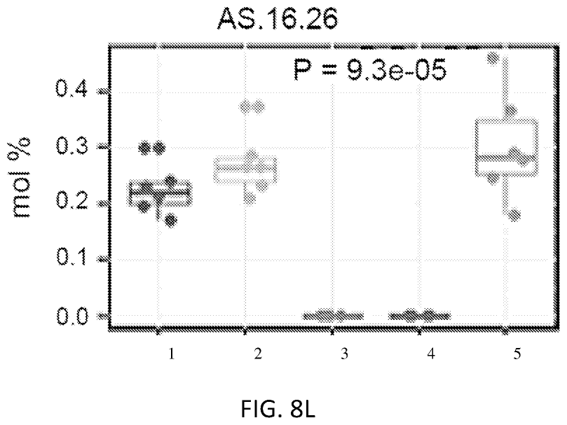

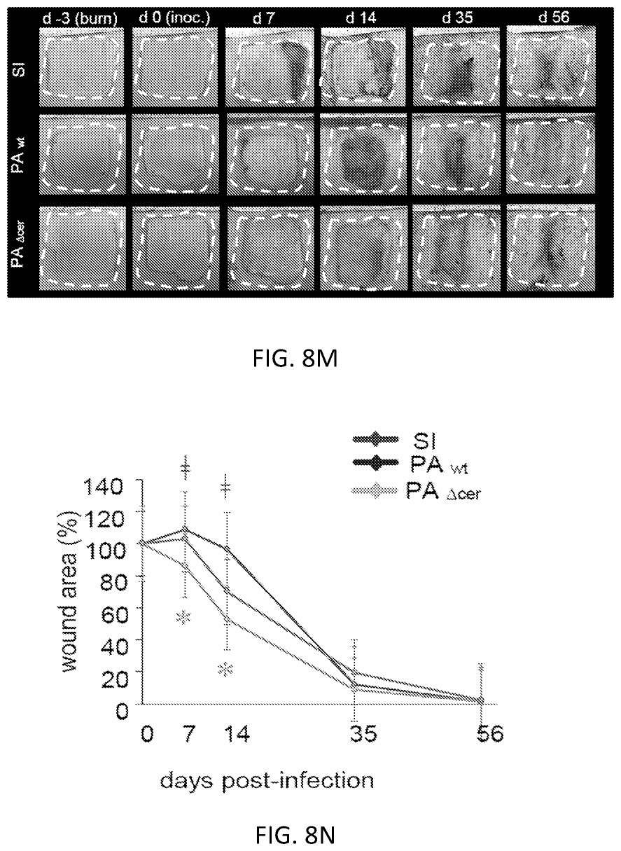

Biofilm-dependent loss of skin ceramide was evident in PAwt, but not in PAΔCer (FIG. 1D). In vitro studies identified that exposure to host skin lipids potently induced bcdase in PAwt; such induction was attenuated in PAwt exposed to depleted skin lipids (FIG. 6E). Inducible bcdase was associated with the induction of the Pseudomonas SphR gene (FIGS. 6F & 6G). SphR is known to function as the transcriptional activator of bcdase. Biofilm-dependent loss of skin ceramides was characterized employing a lipidomics approach. Nineteen long-chain cutaneous ceramides were observed to be depleted in response to biofilm infection by PAwt, but not PAΔCer (FIGS. 1F, 7A-7S). Principal component analyses (PCA) revealed that the abundance of cutaneous ceramides in response to PAwt was statistically distinct from the cluster of cutaneous ceramide levels in response to PAΔCer and sham exposure. Mechanistic studies addressing the loss of keratinocyte ceramide following PAwt biofilm infection were conducted in vitro (FIG. 7T). Ceramidastin, an inhibitor of PA ceramidase, rescued keratinocyte ceramide against loss caused by PAwt biofilm infection (FIG. 7U). Consistent data on rescue of ceramides by ceramidastin was observed in ex vivo studies on extracted skin lipids treated with biofilm-conditioned media (FIGS. 8A-L).

Wounds in all three groups of infection (SI, PAwt and PAΔCer) were studied over a period of 56 days. During this period, all of these wounds were completely closed as evident by wound planimetry (FIG. 8M-N) as well as histology. Skin barrier function, i.e., skin barrier integrity was studied by measuring trans-epidermal water loss (TEWL). Under conditions of PAwt biofilm infection, depletion of skin ceramides was associated with elevated TEWL (FIG. 1F). Such impairment in skin barrier function was not observed in response to PAΔCer (FIG. 1F). These findings establish a causal relationship between induction of bcdase and inability of the repaired skin to restore barrier integrity.

### Skin Lipids Augment Biofilm Via Bcdase.

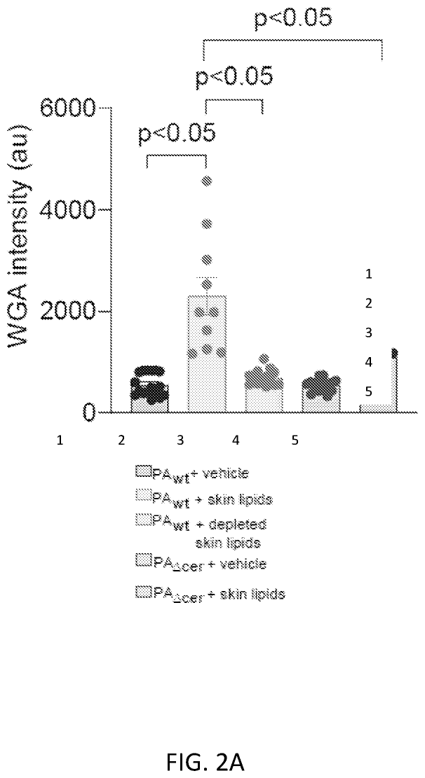

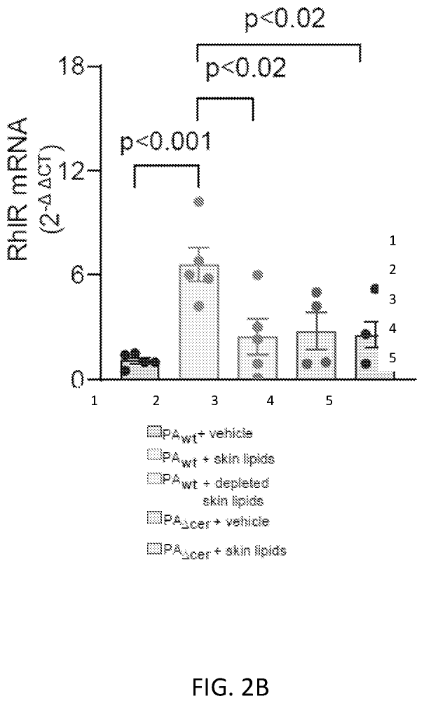

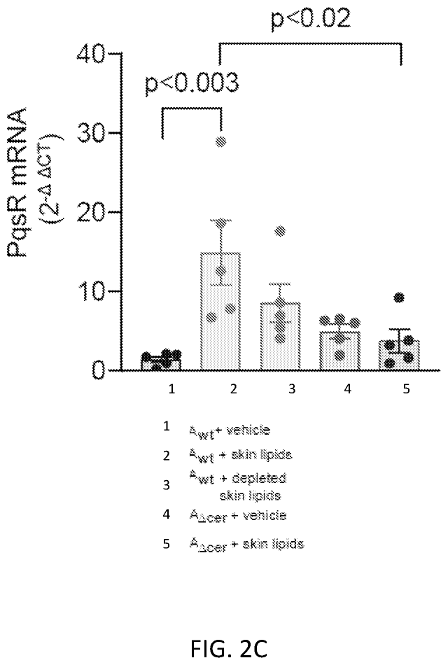

Study of skin tissue extract, native or lipid-depleted (FIGS. 9A-B), demonstrated clear role of lipids in augmenting biofilm formation. To test the significance of breakdown of skin ceramides on biofilm formation, extracted lipids were added to bacteria that were sufficient or deficient in bcdase. Addition of skin lipid extract markedly enhanced biofilm formation in PAwt as measured by SEM and EPS staining. Such augmentation was blunted in PAΔCer. This pointed towards a likely role of skin ceramide degradation products in biofilm formation (FIG. 9C-D). During biofilm formation, bacteria use quorum sensing (QS) to coordinate behaviors such as antibiotic resistance. In PA, QS is driven by a series of small molecule receptors, including the master QS systems pqsR and rh1R26. In line with structural observations on biofilm aggregates, the expression of pqsR, and rh1R, was markedly high in PAwt biofilm treated with host lipids as compared to PAΔCer under the same treatment conditions (FIGS. 2B & 2C). However, no significant difference was observed in the growth rate of PAΔCer and PAwt in their planktonic state in nutrient broth (FIG. 9H). However, increased growth of PAΔCer was observed in presence of host lipids (FIG. 9I). This is in consistent with the in vivo observation where PAΔCer exhibited faster growth. Though, PAwt was a comparative slow grower in presence of host, it exhibited increased biofilm formation as documented through blue phenazine (pyocyanin) formation, WGA staining and increased expression of pqsR gene PqsR/mvfR is known to be required for pyocyanin formation. Pyocyanin contributes to biofilm formation by facilitating extracellular DNA binding to PA28. These data thus demonstrated that the microbial pathogens are not capable of mounting the complex threat in isolation and that they must co-opt host skin lipids to generate complex biofilms.

### Wound Biofilm Downregulates Host Skin CerS3 and Depletes Dihydroceramide.

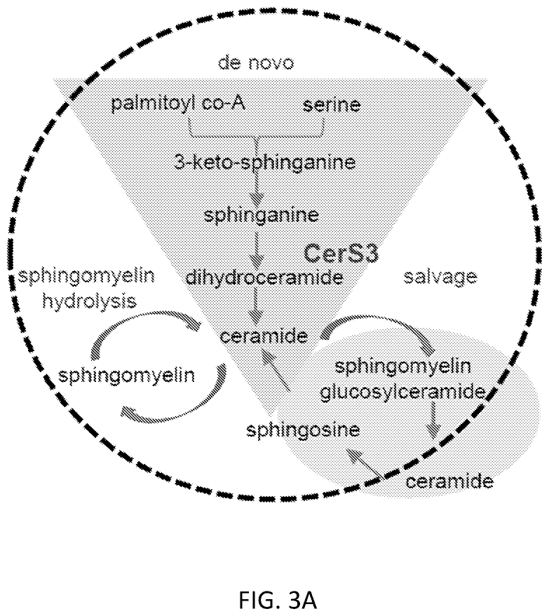

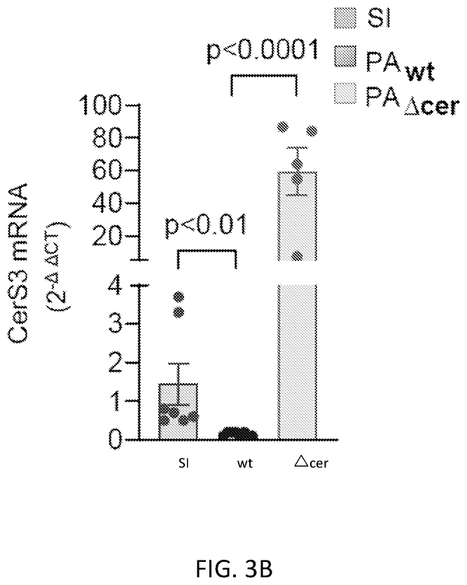

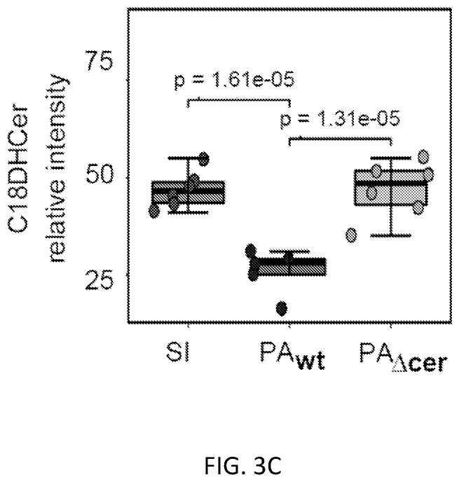

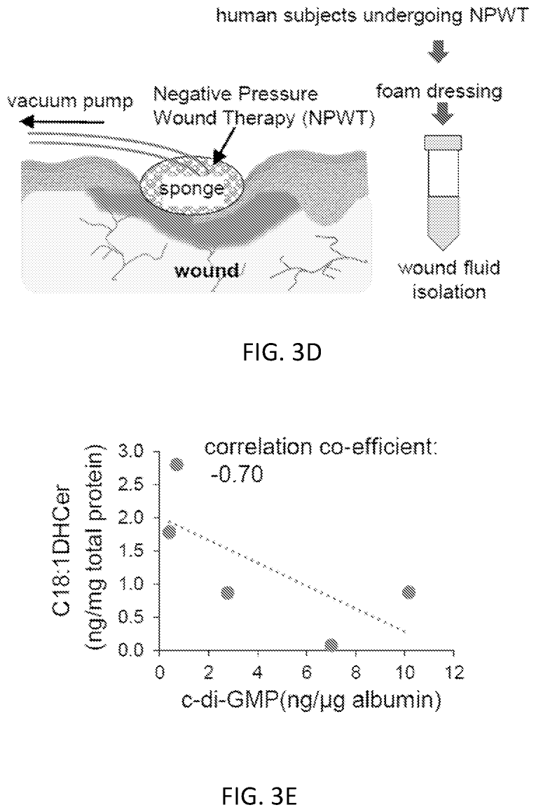

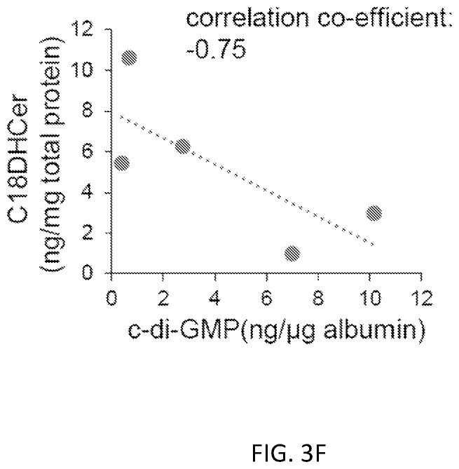

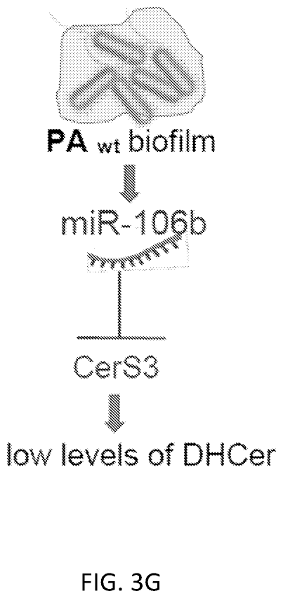

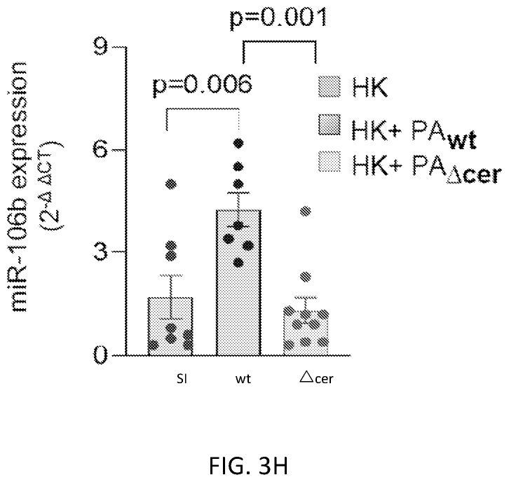

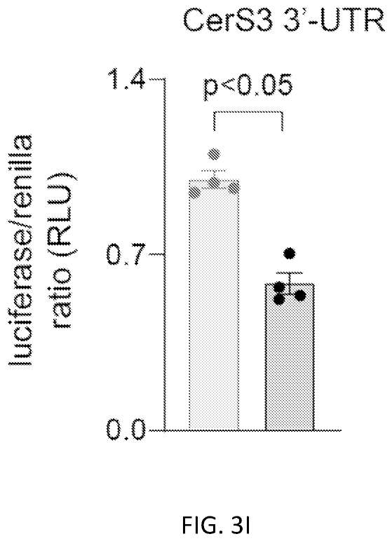

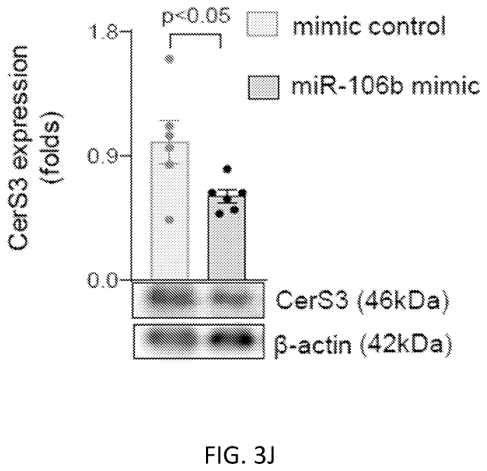

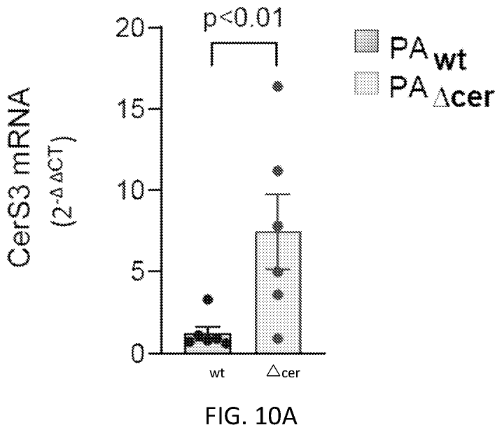

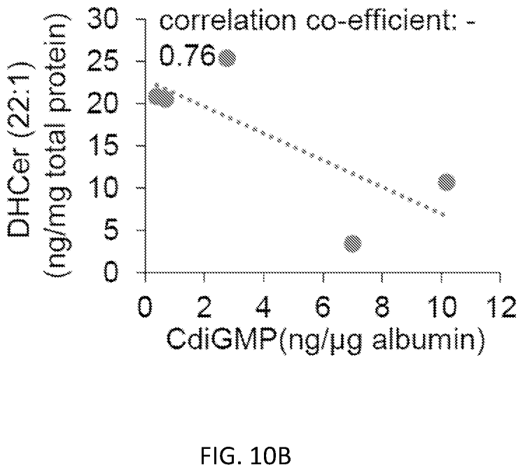

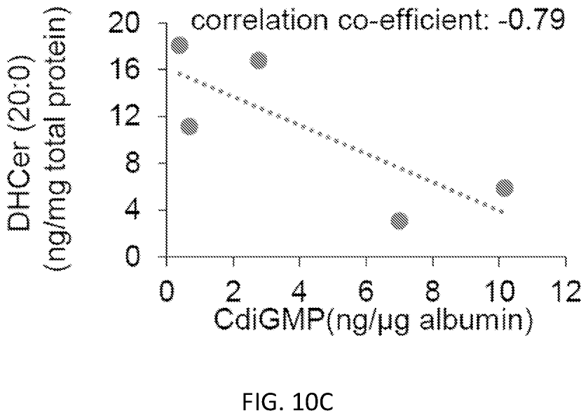

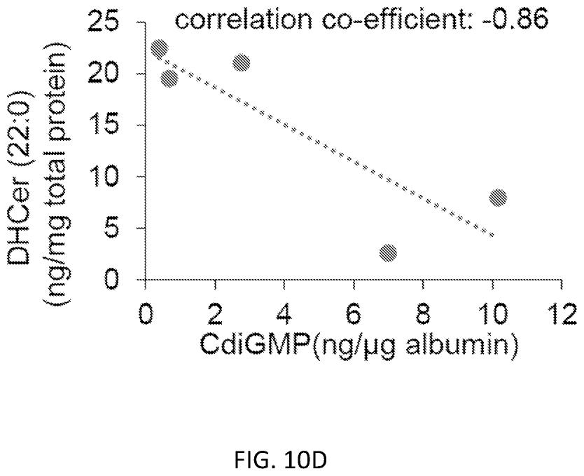

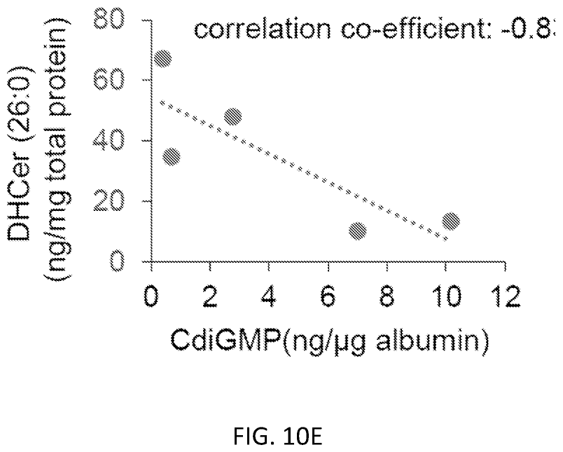

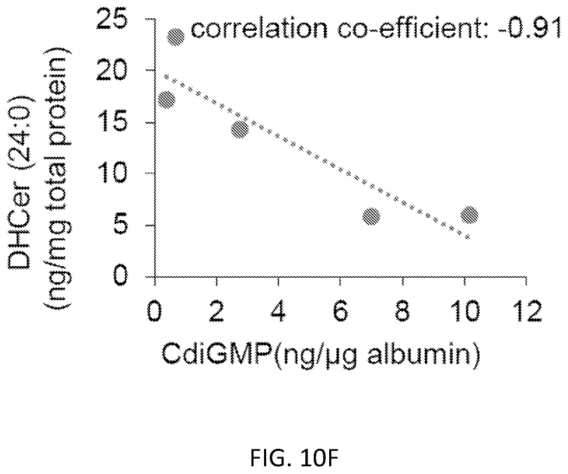

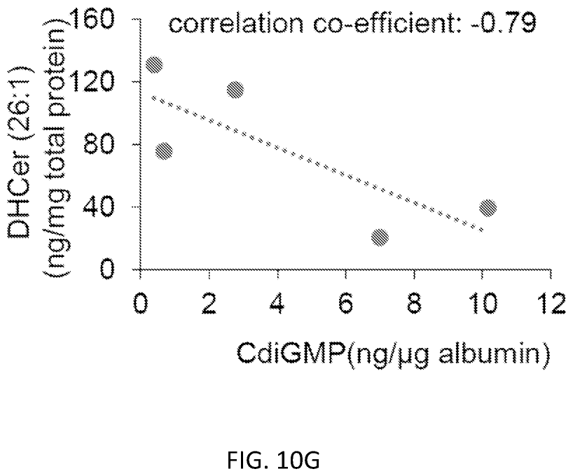

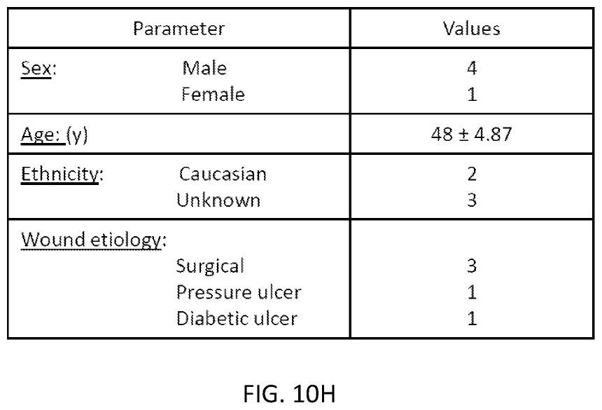

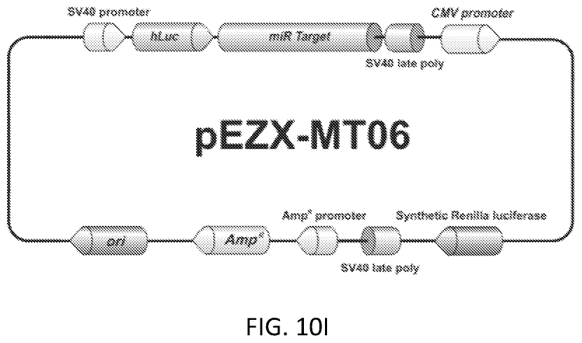

Skin ceramide homeostasis relies on a CerS3-dependent biosynthetic pathway that produces long-chain ceramides (FIG. 3A). Wound biofilm infection compromised ceramide biosynthesis by downregulating CerS3 expression in the PAwt infected wounds, but not in PAΔCer (FIGS. 3B, 10A). Blunted cutaneous CerS3 expression was associated with depletion of long-chain dihydroceramide levels in the porcine skin tissue exposed to PAwt infection (FIG. 3C). To determine the presence of PA biofilm, cyclic di-GMP was used as a surrogate marker in the wound fluids from chronic wound patients. Consistent with our findings from pre-clinical porcine studies, the investigation of wound fluid from chronic wound patients revealed tight correlation between lowering of long-chain dihydroceramide levels with elevated levels of a PA biofilm marker cyclic di-GMP (FIGS. 3E-3F, 10B-10H). Thus, PA biofilms, as marked by diGMP, was associated with lower levels of dihydroceramide.

miR-106b is identified as biofilm-inducible in wound-edge skin tissue with a pathogenic role. miR-106b was induced in response to biofilm infection caused by PAwt, but not by PAΔCer (FIGS. 3G-3H). CerS3 is subject to post-transcriptional gene silencing by miRNA. RNAHybrid™ analyses revealed that the 3′-UTR of CerS3 is likely to be targeted by miR-106b (FIGS. 3I-3J). Biological validation of such prediction was conducted in human keratinocytes. Delivery of miR-106b mimic significantly lowered CerS3 3′-UTR reporter activity (FIGS. 3I, 10I). Consistent with this finding, miR-106b mimic decreased CerS3 protein expression (FIG. 3J).

### Arrest of PPARδ Activity Following Biofilm-Dependent Ceramide Depletion.

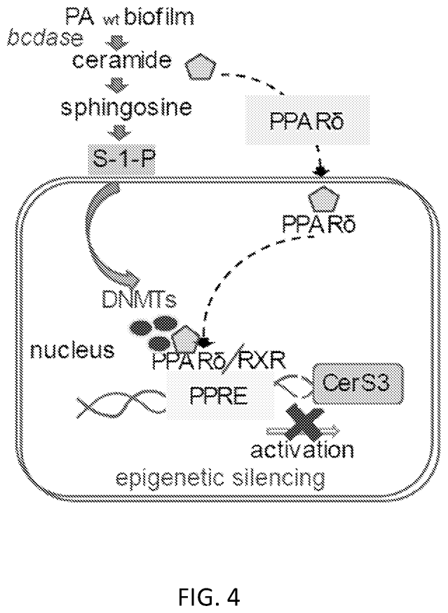

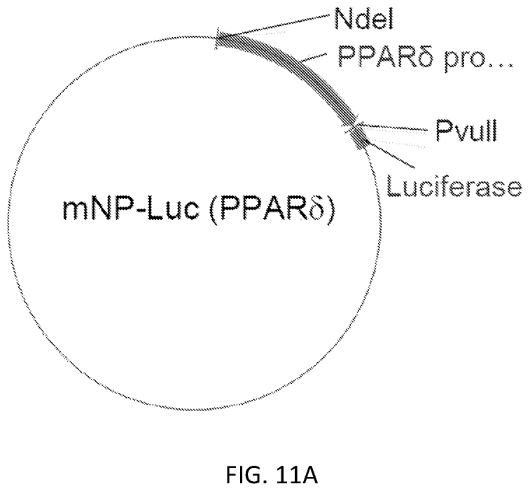

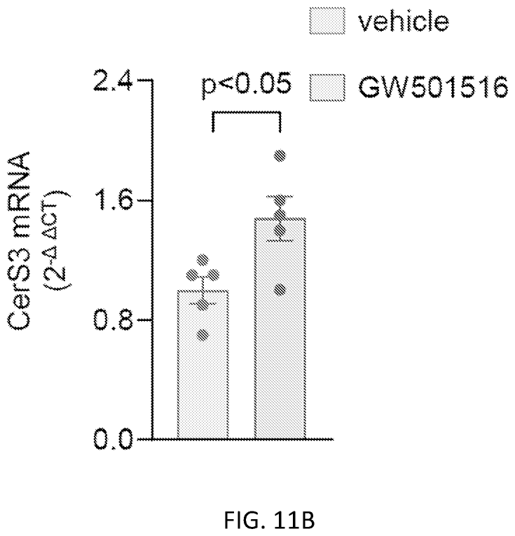

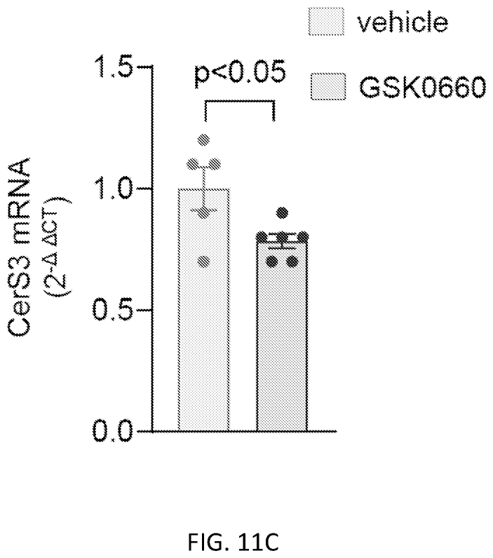

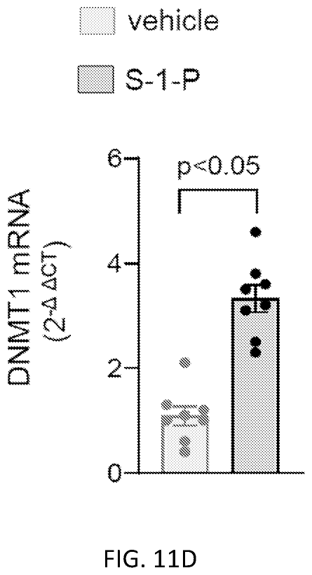

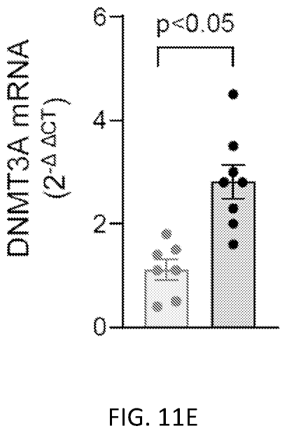

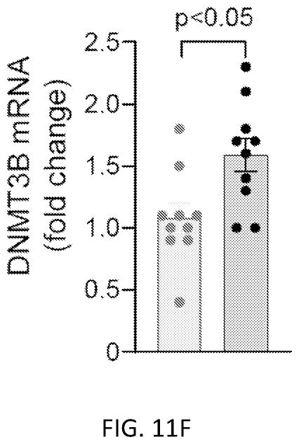

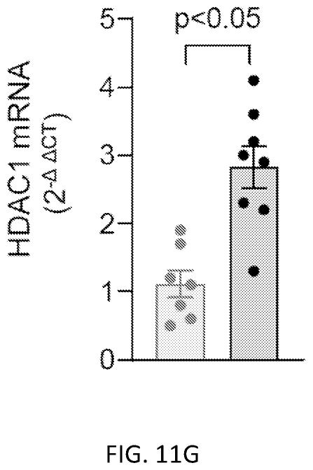

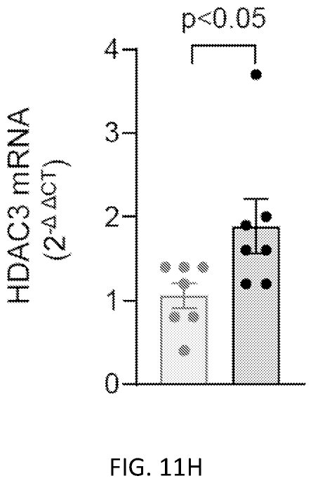

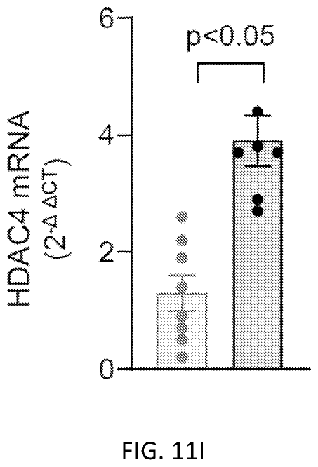

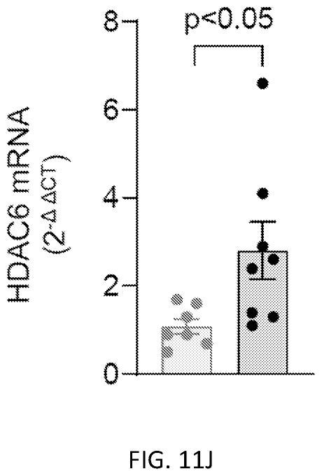

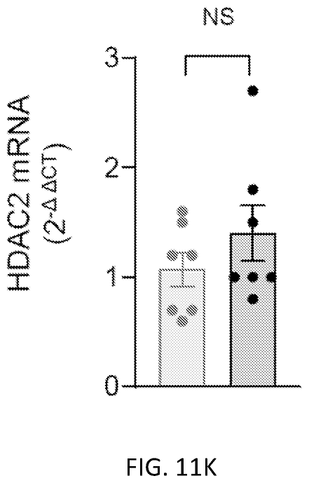

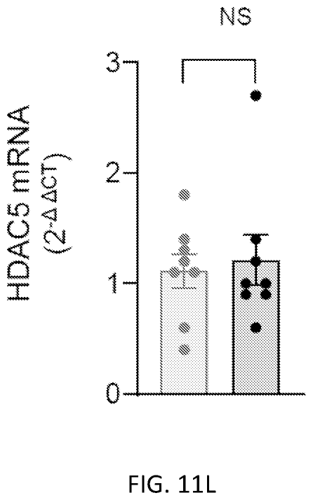

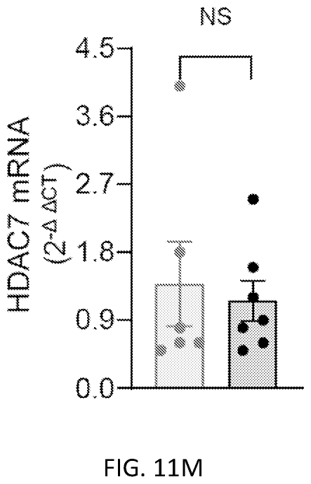

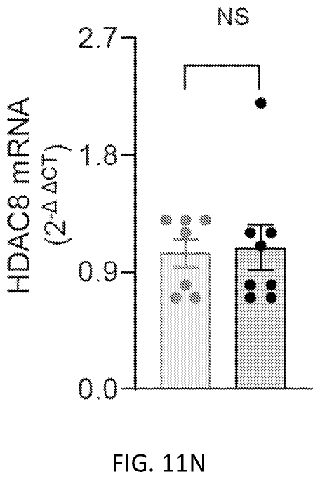

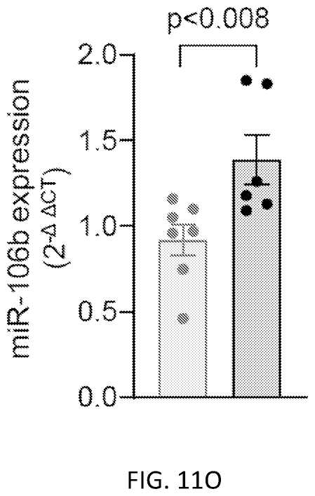

In the peroxisome proliferator-activated receptor (PPAR) family of transcription factors, PPARδ specifically is ceramide-sensitive. In biofilm-affected ceramide-depleted tissue, PPARδ expression was downregulated. Such effect was not observed under conditions of PAΔCer infection pointing towards a causative role of skin ceramide depletion (FIG. 4). To address the underlying mechanisms, studies on human keratinocyte biofilm infection were conducted. Consistent with findings from porcine studies, biofilm infection blunted PPARδ expression. Such effect was rescued in the presence of the PA ceramidase inhibitor, ceramidastin. Skin ceramides are primarily long-chain (≥C18). Thus, C18 and C24 ceramides were tested for their ability to regulate PPARδ function. In human keratinocytes, these long-chain ceramides induced PPARδ transactivation as well as transcription. PPARδ agonist GW501516 increased expression of CerS3 (FIG. 11B) whereas its antagonist GSK0660 blunted the expression of CerS3 (FIG. 11C) in keratinocytes. These findings constitute evidence demonstrating direct regulation of PPARδ by ceramides in keratinocytes.

Spingosine-1-phosphate methylates PPARδ promoter. Cutaneous ceramide is degraded to sphingosine by bacterial ceramidase. Sphingosine is phosphorylated to sphingosine-1 phosphate (S-1-P) which is a bioactive lipid and can epigenetically downregulate gene regulation. Thus, a plausible role of S-1-P in regulating PPARδ in our experimental systems was tested. In the porcine pre-clinical model of wound biofilm infection elevated levels of S-1-P was detected following PAwt, but not PAΔCer, infection. This finding indicated that the detected S-1-P was a breakdown product of skin ceramide acted upon by bcdase. Under standard culture conditions when human keratinocytes were treated with the bioactive sphingolipid S-1-P, gene expression of PPARδ was blunted. To determine whether such downregulation of PPARδ was epigenetically regulated, CpG methylation of the PPARδ promoter was studied.

S-1-P caused promoter methylation. Such increased PPARδ promoter methylation was associated with augmented catalytic activity of DNA methyl transferase 3B (DNMT3B). A survey of the effects S-1-P on epigenetic regulators unveiled broader effects on gene expression favoring DNA methylation and histone deacetylation. Induction of miR-106b by S-1-P constitutes an additional epigenetic mechanism by which S-1-P may attenuate PPARδ (FIG. 11O).

PPARδ is a novel target of biofilm induced miR-106b. The search for PPARδ targeting miRs, that were also biofilm-inducible, led to the identification of miR-106b as a candidate. Delivery of miR-106b mimic compromised PPARδ levels both at mRNA and protein levels. PPARδ was further validated as a target for miR-106b by 3′UTR luciferase reporter assay. Systematic studies thus established PPARδ as a target of biofilm inducible miR-106b in keratinocytes.

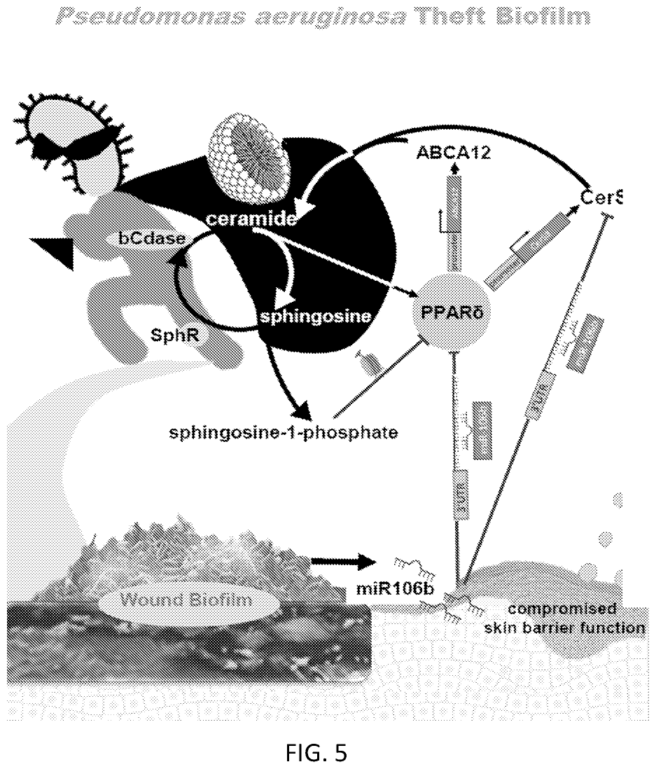

Epidermal lipid transporter ABCA12 is compromised following wound biofilm infection. Skin ceramides are responsible for an estimated 50% of all cutaneous lipids. Other skin lipids play a significant role in enabling skin barrier function. These two are known to interactively maintain skin health. ABCA12, the transcription of which is PPARS-dependent (FIGS. 12A &12B), is an epidermal keratinocyte lipid transporter. Wound-site ABCA12 expression was attenuated in response to biofilm infection by PAwt, but not SI or PAΔCer. Such downregulation of ABCA12 was associated with overt changes in skin lipid distribution. At the wound-site epidermis, levels of polar and neutral lipids were sharply lower in response to biofilm infection by PAwt, but not by SI or PAΔCer. Mechanistic follow up studies on human keratinocytes revealed that loss of ABCA12 in response to biofilm infection by PAwt was prevented by ceramidastin demonstrating that the loss was caused by a breakdown product of keratinocyte ceramide acted upon by bcdase. Such finding was consistent with the prevention of loss of ABCA12 in response to infection by PAΔCer. FIG. 5 presents a mechanistic summary.

Armed with a wide range of lipid metabolizing inducible enzymes, PA is known to exploit host lipids to facilitate host cell binding and to evade host immune defenses. In macrophages of the innate immune defense system, PA bolsters acid sphingomyelinase activity causing release of host lipid ceramides producing sphingolipid-rich rafts which helps internalization of PA. In the lungs, PA lipoxygenase (pLoxA) oxidize host arachidonic acid-phosphatidylethanolamine to cause bronchial epithelial ferroptosis and establish airway biofilm. The notion that host lipids may be pre-emptively primed to compromise the ability of pathogenic microbes to co-opt them emerges as result. Induction of bacterial ceramidases by three orders of magnitude in PA biofilm-infected wound tissue called for a systematic investigation testing its significance in the healing response in biofilm affected wounds.

The Wound Healing Society recommends the study of porcine model as the most relevant preclinical model of skin wound healing. The current work is based on the study of an established model of chronic wound biofilm infection in immune-competent pigs in vivo. The approach results in the establishment of an induced polymicrobial wound biofilm comprising of Pseudomonas aeruginosa (PA) and Acinetobacter baumannii. Non-infected skin wounds colonized by normal skin flora of the porcine served as baseline control and were referred to as spontaneous infection (SI). In this model, those skilled in the art understand that PA establishes itself as the dominant strain as the wound becomes chronic.

Skin serves the primary function of affording barrier defense. Loss of skin barrier increases vulnerability to infection and allergens. Compromised skin barrier function is also associated with atopic dermatitis, psoriasis, contact dermatitis, and some specific genetic disorders. Central to enabling the barrier function of skin are the skin ceramides. Ceramide homeostasis of the skin depends on a dynamic balance between biosynthetic and catabolic pathways. The observation that biofilm infection by PAwt, but not PAΔCer, compromises barrier function of the repaired skin provides direct evidence implicating ceramide depletion in impaired restoration of barrier function during healing. CerS3 is recognized as the primary catalyst of long chain cutaneous ceramide synthesis. Biofilm-inducible miR-106b is recognized as a post-transcriptional gene silencer of CerS3.

PA leverages host lipids to bolster biofilm formation. Addition of cutaneous porcine lipid augmented biofilm formation in a manner sensitive to delipidation. Lipids per se were not the trigger because such augmentation of biofilm was absent with PAΔCer. Biofilm formation is linked with quorum sensing (QS) pathway of PA. The current work demonstrates that ceramide breakdown products are capable of inducing SphR. During antibiotic resistance of PA, SphR is involved in quorum sensing VqSM-SphR interaction. This pathway represents a plausible mechanism by which skin lipids may induce QS. In transcriptional regulation of host skin lipids, peroxisome proliferator-activated receptor PPARδ plays a central role. This hub emerged as a central player in the paradigm unveiled by the findings disclosed herein. Basal PPARδ activity in the skin is driven by ceramides. The PA biofilm dependent amplification loop, as described above, depleted skin ceramides thus lowering PPARδ activity. As part of that loop as ceramides were catabolized to sphingosine, S-1-P was produced. S-1-P epigenetically silenced PPARδ expression. In lung injury secondary to inflammation caused by P. aeruginosa, ceramide-derived sphingosine and S1P are directly implicated. Interestingly, ceramide can also act as antimicrobials. Sphingosine effectively killed S. aureus, Streptococcus pyogenes, Micrococcus luteus, Propionibacterium acnes, Staphylococcus epidermidis and moderately killed P. aeruginosa. Sphingosine prevented and eliminated Staphylococcus epidermidis biofilm on orthopedic implant materials. Sphingosine binds to bacterial membrane cardiolipin and limit growth. Bacterial growth retardation is inherent to biofilm formation. Elevated S-1-P, a derivative of sphingosine, is associated with higher biofilm formation.

A third mechanism to down-regulate PPARδ was contributed by PA biofilm inducible miR-106b. This may be viewed as a well-coordinated effort by PA biofilm to disable skin PPARδ and therefore hijack host metabolic processes to augment biofilm fate. Of particular interest is the observation that this entire cascade of events is triggered by host lipids. As it relates to the functional significance of PPARδ in barrier function of the skin, it is known that topical application of an agonist of PPARδ accelerates restoration of such function following injury.

The ATP-binding cassette (ABC) transporter ABCA12, transcriptionally regulated by PPARδ, encodes a highly conserved group of proteins involved in active transport of a variety of lipids across biological membranes. In epidermal keratinocytes, PPARδ upregulates ABCA12. Loss of skin wound-site PPARδ in response to PA biofilm infection was associated with compromised ABCA12 expression. At the wound-site, where covering of the defect with repaired skin has been achieved it was noted that this ABCA12-deficient epidermis was also compromised in abundance of lipids. Such pathological manifestation has been also reported in congenital ichthyoses where low ABCA12 is associated with compromised skin barrier. This is consistent with demonstrations that wounds with a history of biofilm infection appears closed but are not functionally closed as the site is deficient in barrier function. Closed wound-site with a history of biofilm infection, featuring compromised barrier function, is known to biomechanically deficient with weak tensile strength. This observation, taken together with the report that skin deficient in barrier function act as window allowing entry of pathogenic allergens to the body, points towards the understanding that such affected site may be prone to wound recidivism and other threats to general health.

In the setting of cutaneous wounds, pathogenic PA biofilm formation relies on the theft of host lipid factors which the bacteria use to turn on and to sustain its bolstered ceramidase system which is otherwise weak. PA biofilm formation was highly responsive to its microenvironment such that in the context of skin wounds it utilized ceramide breakdown products to augment biofilm aggregates. This process was initiated by a massive induction of bacterial ceramidase in response to host lipids. Downstream products of such metabolism such as sphingosine and S-1-P were directly implicated in induction of ceramidase and inhibition of PPARδ, respectively. PA biofilm also silenced PPARδ via induction of miR-106b. Low PPARδ limits ABCA12 expression resulting in disruption of skin lipid homeostasis. Barrier function of the skin was thus compromised. The significance of such defect in the functional deficiency of the skin with respect to risk of infection and wound recurrence warrant further consideration.

Aspects of the present disclosure can be described as embodiments in any of the following enumerated clauses. It will be understood that any of the described embodiments can be used in connection with any other described embodiments to the extent that the embodiments do not contradict one another.

Clause 1. A composition comprising: a therapeutically effective amount of a bacterial ceramidase inhibitor and a ceramide.

Clause 2. A composition according to preceding clause 1, where the composition is a skin care composition.

Clause 3. A composition according to any of the preceding clauses, where the composition is a pharmaceutical composition.

Clause 4. A composition according to any of the preceding clauses, where the composition is formulated to be a controlled release composition.

Clause 5. A composition according to any of the preceding clauses, where at least one component is microencapsulated.

Clause 6. A composition according to any of the preceding clauses, where the bacterial ceramidase inhibitor obtained from a microorganism.

Clause 7. A composition according to any of the preceding clauses, where the bacterial ceramidase inhibitor is ceramidastin.

Clause 8. A composition according to any of the preceding clauses, where the ceramide is hydroxypropyl bispalmitamide MEA.

Clause 9. A composition according to any of the preceding clauses, further comprising wound healing agents.

Clause 10. A composition according to any of the preceding clauses, further comprising one or more excipients acceptable for skin.

Clause 11. A composition according to any of the preceding clauses, further comprising a sterol.

Clause 12. A composition according to any of the preceding clauses, further comprising a sterol that is cholesterol.

Clause 13. A composition according to any of the preceding clauses, further comprising purified water.

Clause 14. A composition according to any of the preceding clauses, further comprising a fatty acid that is conjugated linoleic acid and/or glyceryl stearate.

Clause 15. A composition according to any of the preceding clauses, further comprising a fatty compound that is squalene.

Clause 16. A composition according to any of the preceding clauses, further comprising a polyol that is glycerin.

Clause 17. A composition according to any of the preceding clauses, further comprising an emulsifier that is PEG-100 stearate and/or phenoxyethanol.

Clause 18. A composition according to any of the preceding clauses, further comprising a silicone oil that is dimethicone.

Clause 19. A composition according to any of the preceding clauses, further comprising a preservative selected from the group consisting of disodium EDTA, sorbic acid, and citric acid.

Clause 20. A composition according to any of the preceding clauses, further comprising a pH adjuster selected from the group consisting of citric acid and potassium hydroxide.

Clause 21. A composition according to any of the preceding clauses, further comprising a thickening agent that is xanthan gum.

Clause 22. A composition according to any of the preceding clauses, where the composition is a topical formulation selected from a cream, lotion, serum, balm, gel for topical application, granule, powder, paste, liquid formulation, syrup, solution, emulsion, and a suspension.

Clause 23. A composition according to any of the preceding clauses, where one or more components of the composition is microencapsulated.

Clause 24. A method to treat a skin condition in a mammal in need thereof, the method comprising the step of: administering a therapeutically effective amount of a composition comprising at least one of a bacterial ceramidase inhibitor and a ceramide to the skin condition in the mammal in need thereof.

Clause 25. A method according to preceding clause 24, where the administering step comprises a topically administration of the composition.

Clause 26. A method according to any of preceding clauses 24-25, where the skin condition is ageing skin.

Clause 27. A method according to any of preceding clauses 24-25, where the skin condition is diabetic skin.

Clause 28. A method according to any of preceding clauses 24-25, where the skin condition is a wound.

Clause 29. A method according to any of preceding clauses 24-25, where the skin condition is a wound on a lower extremity of the mammal.

Clause 30. A method according to any of preceding clauses 24-25, where the skin condition is a chronic wound.

Clause 31. A method according to any of preceding clauses 24-25, where the skin condition is an open wound.

Clause 32. A method according to any of preceding clauses 24-25, where the open wound includes a polymicrobial biofilm infection.

Clause 33. A method according to any of preceding clauses 24-32, where the open wound includes reduced ceramides.

Clause 34. A method according to any of preceding clauses 24-33, where the open wound includes a biofilm infection comprising Pseudomonas aeruginosa.

Clause 35. A method according to any of the preceding clauses 24-34, where the open wound is a burn.

Clause 36. A method according to any of preceding clauses 24-35, where the skin condition is a closed wound.

Clause 37. A method according to clause 36, where the closed wound is a burn.

Clause 38. A method according to any of the preceding clauses 35-36, where the skin condition is a closed wound that has been closed for less than 2 weeks.

Clause 39. A method according to any of preceding clauses 24-38, where the mammal is selected from the group consisting of humans, primates, rats, mice, rabbits and guinea pigs, dogs, cats, and horses.

Clause 40. A method according to any of preceding clauses 24-39, where the mammal is a human.

Clause 41. A method according to any of preceding clauses 24-40, where the administered composition increases skin barrier function in the treated skin.

Clause 42. A method according to any of preceding clauses 24-41, where the administered composition reduces trans epidermal water loss in the treated skin.

Clause 43. A method according to any of preceding clauses 24-42, where the administered composition increases skin barrier function in the treated skin and the skin barrier function is skin barrier integrity.

Clause 44. A method according to any of preceding clauses 24-43, where the bacterial ceramidase inhibitor is administered in an amount ranging from about 0.001% to about 10% by weight with respect to total weight of a composition.

Clause 45. A method according to any of preceding clauses 24-44, where the bacterial ceramidase inhibitor is administered in an amount ranging from about 0.01% to about 5% by weight with respect to total weight of a composition.

Clause 46. A method according to any of preceding clauses 24-45, where the bacterial ceramidase inhibitor is administered in an amount ranging from about 0.1% to about 0.5% by weight with respect to total weight of a composition.

Clause 47. A method according to any of preceding clauses 24-46, where the ceramide is administered in an amount ranging from about 0.001% to about 10% by weight with respect to total weight of a composition.

Clause 48. A method according to any of preceding clauses 24-47, where the ceramide is administered in an amount ranging from about 0.01% to about 5% by weight with respect to total weight of a composition.

Clause 49. A method according to any of preceding clauses 24-48, where the ceramide is administered in an amount ranging from about 0.1% to about 0.5% by weight with respect to total weight of a composition.

Clause 50. A method according to any of preceding clauses 24-49, where negative pressure wound therapy accompanies the treatment.

Clause 51. A composition comprising
an inhibitor of microbial pathogen lipase activity;
an epidermal lipid, selected from the group consisting of ceramide, phospholipids, sphingolipids, cholesterol, free fatty acids, and triglycerides; and
a pharmaceutically acceptable carrier.

Clause 52. The composition of clause 51 further comprising any of the components of clauses 1-23 either individually or in combination.

Clause 53. The composition of clause 51 or 52 further comprising an anti-microbial agent, optionally wherein the anti-microbial agent is an antibiotic.

Clause 54. The composition of any one of clauses 51-53 wherein the epidermal lipid is a ceramide having the general structure of

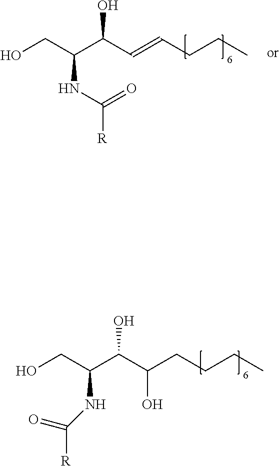

wherein R is —(CH₂)₁₈₋₂₉(CH₃), —(CHOH)(CH₂)₁₈₋₂₉(CH₃) or —(CH₂)₁₈₋₃₀O(CO)(CH₂)₇(CH═CH)CH₂(CH═CH)(CH₂)₄CH₃.

Clause 55. The composition of any one of clauses 51-54 wherein the ceramidase inhibitor is Ceramidastin or hydroxypropyl bispalmitamide MEA.

Clause 56. The composition of any one of clauses 51-55 wherein the composition is formulated as a cream, ointment, balm, lotion or paste for topical application.

Clause 57. The composition of any one of clauses 1-55 wherein the ceramidase inhibitor is selected from any known ceramidase inhibitor, including those disclosed in published US application nos. 20180369211 and 20110251197, the disclosures of which are expressly incorporated herein.

Clause 58. A method of treating a skin condition associated with compromised barrier function in a mammal in need thereof, the method comprising the step of: administering a therapeutically effective amount of any of the compositions of clauses 1-23 and 51-57 to said mammal.

Clause 59. The method of clause 58 wherein the skin condition is a wound and the administration step comprises topically apply said composition to the wound, optionally wherein the wound is infected with a microbial pathogen, optionally wherein the microbial pathogen is Pseudomonas aeruginosa.

Clause 60. A method of treating a wound at risk of infection, or infected, by a bacterial pathogen, said method comprising contacting tissues associated with said wound with a therapeutically effective amount of any of the compositions of clauses 1-23 and 51-57.

Clause 61. The method of clause 60 wherein tissues associated with said wound are contacted with a topically applied composition, optionally wherein the wound is infected with a microbial pathogen, optionally wherein the microbial pathogen is Pseudomonas aeruginosa.

Other variations or embodiments will be apparent to a person of ordinary skill in the art from the above-description. Thus, the foregoing embodiments are not to be construed as limiting the scope of the claimed invention. All references disclosed are expressly incorporated by reference in in their entirety.

1. A composition comprising
an inhibitor of microbial lipase activity;
an epidermal lipid, selected from the group consisting of ceramide, phospholipids, sphingolipids, cholesterol, free fatty acids, and triglycerides; and
a pharmaceutically acceptable carrier.

2. The composition of claim 1 wherein the inhibitor is a ceramidase inhibitor.

3. The composition of claim 1 further comprising an anti-microbial agent.

4. The composition of claim 3 wherein the anti-microbial agent is an antibiotic.

5. The composition of claim 1 wherein the epidermal lipid is a ceramide, optionally a long-chain dihydroceramide.

6. The composition of claim 5 wherein the ceramide has the general structure of 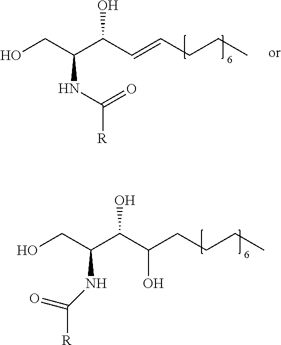
wherein R is —(CH₂)₁₈₋₂₉(CH₃), —(CHOH)(CH₂)₁₈₋₂₉(CH₃) or —(CH₂)₁₈₋₃₀O(CO)(CH₂)₇(CH═CH)CH₂(CH═CH)(CH₂)₄CH₃.

7. The composition of claim 6 wherein the ceramidase inhibitor is Ceramidastin.

8. The composition of claim 7 formulated as a cream for topical application.

9. A method of treating a skin condition in a mammal in need thereof, the method comprising the step of: administering a therapeutically effective amount of a composition of claim 1 to said mammal.

10. The method of claim 9 wherein the skin condition is a wound and the administration step comprises topically applying said composition to the wound.

11. The method of claim 9 wherein the skin condition is compromised barrier function and the administration step comprises topically applying said composition to the wound.

12. The method of claim 10 wherein the wound is infected with a microbial pathogen.

13. The method of claim 12 wherein the microbial pathogen is Pseudomonas aeruginosa.

14. A method of treating a wound at risk of infection, or infected, by a bacterial pathogen, said method comprising contacting tissues associated with said wound with a composition comprising
a therapeutically effective amount of a ceramidase inhibitor; and
one or more ceramides.

15. The method of claim 14 wherein the ceramidase inhibitor is Ceramidastin.

16. The method of claim 14 wherein the ceramide has the general structure of 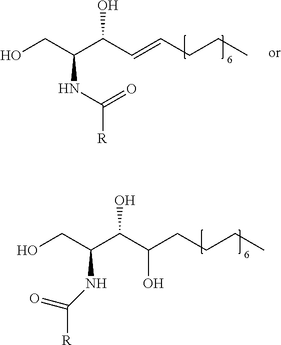
wherein R is —(CH₂)₁₈₋₂₉(CH₃), —(CHOH)(CH₂)₁₈₋₂₉(CH₃) or —(CH₂)₁₈₋₃₀O(CO)(CH₂)₇(CH═CH)CH₂(CH═CH)(CH₂)₄CH₃.
